(19) United States
(12) Patent Application Publication
(10) Pub. No.: US 2016/0367144 A1
(43) Pub. Date: Dec. 22, 2016

(54) PHOTODETECTION UNIT AND BIOLOGICAL INFORMATION DETECTION APPARATUS

(71) Applicant: SEIKO EPSON CORPORATION, Tokyo (JP)

(72) Inventor: Atsushi MATSUO, Azumino-shi (JP)

(73) Assignee: SEIKO EPSON CORPORATION, Tokyo (JP)

(21) Appl. No.: 15/117,718

(22) PCT Filed: Mar. 2, 2015
(86) PCT No.: PCT/JP2015/001083

§ 371 Date: Aug. 9, 2016

(30) Foreign Application Priority Data
Mar. 13, 2014 (JP) 2014-050080
Mar. 27, 2014 (JP) 2014-065982

(51) Int. Cl. A61B 5/00

(52) U.S. Cl. CPC A61B 5/0059; A61B 5/681; A61B 5/7207; A61B 5/7455; A61B 2562/0238

(57) ABSTRACT

A photodetection unit includes a substrate, a light emitting part that outputs light to an object, and a light receiving part attached to the substrate and receiving light from the object, wherein a hole part is provided in the substrate, and the light emitting part is attached to the hole part of the substrate.

### CROSS-REFERENCE

This application contains the contents of Japanese Patent Application 2014-050080 filed on Mar. 13, 2014 and Japanese Patent Application 2014-065982 filed on Mar. 27, 2014.

### TECHNICAL FIELD

The present invention relates to a photodetection unit, a biological information detection apparatus, etc.

### BACKGROUND ART

In related art, biological information detection apparatuses that detect biological information including pulse wave etc. of humans are known. PTL 1 and PTL 2 disclose the related art of pulsimeters as examples of the biological information detection apparatuses. The pulsimeter is attached to e.g., an arm, a wrist, a finger, or the like, and detects pulsation derived from heartbeats and measures the pulse rate.

The pulsimeters disclosed in PTL 1 and PTL 2 are photoelectric pulsimeters and their photodetection units each has a light emitting part that emits light toward a subject as an object and a light receiving part that receives the light from the subject (light having biological information). In the pulsimeter, changes in blood flow are detected as changes in amounts of received light, and thereby, detects the pulse wave. PTL 1 discloses the pulsimeter of a type attached to a wrist and PTL 2 discloses the pulsimeter of a type attached to a finger. Further, PTL 3 discloses an optical sensor in which a light shielding member is provided for a light receiving part.

### CITATION LIST

### Patent Literature

PTL 1: JP-A-2011-139725

PTL 2: JP-A-2009-201919

PTL 3: JP-A-6-273229

### SUMMARY OF INVENTION

### Technical Problem

In the detection apparatuses of biological information etc., the light emitting part of the light detection unit outputs light to an object and various kinds of information is detected based on detection signals obtained by the light receiving part receiving the light from the object. Accordingly, improvement in signal quality of detection signals is an important challenge.

### Solution to Problem

An aspect of the invention relates to a photodetection unit including a substrate, a light emitting part that outputs light to an object, and a light receiving part attached to the substrate and receiving light from the object, wherein a hole part is provided in the substrate, and the light emitting part is attached to the hole part of the substrate.

Another aspect of the invention relates to a photodetection unit including a light emitting part that outputs light to an object, a light receiving part that receives light from the object, a first substrate on which the light emitting part is mounted, and a second substrate on which the light receiving part is mounted, wherein, supposing that a height of the first substrate from a reference surface set at an opposite side to the object with respect to the first substrate is h4 and a height of the second substrate from the reference surface is h5, h5>h4 is satisfied.

Still another aspect of the invention relates to a photodetection unit including a light emitting part that outputs light to an object, a light receiving part that receives light from the object, a first substrate on which the light emitting part is mounted, and a second substrate on which the light receiving part is mounted, wherein, in a state in which an apparatus including the photodetection unit is attached to a subject as the object, supposing that a distance from the first substrate to the object is d1 and a distance from the second substrate to the object is d2, d1>d2 is satisfied.

Yet another aspect of the invention relates to a biological information detection apparatus including the photodetection unit described above.

### BRIEF DESCRIPTION OF DRAWINGS

The invention will be described with reference to the accompanying drawings, wherein like numbers reference like elements.

FIG. 1A shows a comparative example to a technique of an embodiment.

FIG. 1B shows a comparative example to a technique of an embodiment.

FIG. 1C is a side view showing configuration examples of a photodetection unit of the embodiment.

FIG. 1D is a side view showing configuration examples of a photodetection unit of the embodiment.

FIG. 2 is a perspective view showing a configuration example of the photodetection unit of the embodiment.

FIG. 3 is a diagram for explanation of differences in detection signal level of the photodetection unit with or without a lens part.

FIG. 4A is a plan view showing a configuration example of the photodetection unit of the embodiment.

FIG. 4B is a plan view showing a configuration example of the photodetection unit of the embodiment.

FIG. 4C is a side view showing configuration example of the photodetection unit of the embodiment.

FIG. 4D is a plan view showing a configuration example of the photodetection unit of the embodiment.

FIG. 4E is a side view showing a configuration examples of the photodetection unit of the embodiment.

FIG. 5A is another side view showing a configuration example of the photodetection unit of the embodiment.

FIG. 5B is another side view showing a configuration example of the photodetection unit of the embodiment.

FIG. 6 shows a plan view, a side view, a front view, and rear view showing detailed shapes of a light shielding member.

FIG. 7 is an explanatory diagram of heights of a light emitting part, a light receiving part, and a light shielding wall.

FIG. 8 shows a relationship between a distance between the light emitting part and the light receiving part and signal intensity of a detection signal.

FIG. 9 is an explanatory diagram of a relationship between the distances between the light emitting part and the light receiving part and measured distances in a depth direction.

FIG. 10A is an external view of a biological information detection apparatus of the embodiment.

FIG. 10B is an external view of a biological information detection apparatus of the embodiment.

FIG. 11 is an external view of the biological information detection apparatus of the embodiment.

FIG. 12 is an explanatory diagram of attachment of a biological information detection apparatus and communication with a terminal device.

FIG. 13 is a functional block diagram of the biological information detection apparatus.

FIG. 14A is an explanatory diagram of sensor units.

FIG. 14B is an explanatory diagram of sensor units.

FIG. 14C is an explanatory diagram of sensor units.

FIG. 15 is a diagram for explanation of heights from a reference surface.

FIG. 16 is a diagram for explanation of shielding of direct light by a second substrate.

FIG. 17 is a diagram for explanation of distances from first and second substrates to a subject.

FIG. 18 shows an example in which the first and second substrates are integrally formed using a flexible board.

FIG. 19A is a side view of the photodetection unit in which a second light receiving part for noise detection is provided in the first substrate or the second substrate.

FIG. 19B is a side view of the photodetection unit in which a second light receiving part for noise detection is provided in the first substrate or the second substrate.

FIG. 20 is an exploded view showing an internal structure etc. of a case unit.

FIG. 21 is an exploded view showing an internal structure etc. of the case unit.

### DESCRIPTION OF EMBODIMENTS

According to some aspects of the invention, a photodetection unit, a biological information detection apparatus, etc. having higher sensitivity and smaller sizes because of appropriate attachment of light emitting parts to substrates.

One embodiment of the invention relates to a photodetection unit including a substrate, a light emitting part that outputs light to an object, and a light receiving part attached to the substrate and receiving light from the object, wherein a hole part is provided in the substrate, and the light emitting part is attached to the hole part of the substrate.

In the one embodiment of the invention, in the photodetection unit having the light emitting part and the light receiving part, the light emitting part is attached to the hole part provided in the substrate. Thereby, even in the case where the size of the light emitting part is larger or the like, it may be possible to downsize the photodetection unit.

In the embodiment, the light receiving part may be attached to a first surface of the substrate, and the light emitting part may be inserted into the hole part from a second surface side of the substrate as a rear surface of the first surface.

Thereby, it may be possible to efficiently downsize the photodetection unit using the thickness of the substrate.

In the embodiment, the light emitting part may have a lens part that condenses light, and, in the light emitting part, the lens part may be inserted into the hole part to project toward the first surface side.

Thereby, it may be possible to efficiently downsize the photodetection unit using the thickness of the substrate.

In the embodiment, the light emitting part may have a light emitting device, an enclosing part in which the light emitting device is enclosed, and a base part as a base of the enclosing part, wherein the base part may be provided at the second surface side in a state in which the light emitting part is inserted into the hole part.

Thereby, it may be possible to appropriately fix the light emitting part to the substrate while realizing downsizing.

In the embodiment, the base part may have a terminal electrically connected to the light emitting device, and the terminal may be electrically connected to a wire provided on the second surface of the substrate.

Thereby, it may be possible to appropriately connect the light emitting part to the substrate while realizing downsizing.

In the embodiment, a light shielding member that shields at least the light receiving part from light may be provided on the substrate.

Thereby, it may be possible to suppress incidence of light causing noise to the light receiving part.

In the embodiment, the light shielding member may have a light shielding wall that shields light from the light emitting part entering the light receiving part, and, supposing that a height of the light shielding wall is h1 and a height of the light emitting part is h2, h1>=h2 may be satisfied.

Thereby, it may be possible to shield direct light from the light emitting part to the light receiving part using the light shielding wall.

In the embodiment, supposing that a height of the light receiving part is h3, h1>=h2>h3 may be satisfied.

Thereby, it may be possible to shield direct light from the light emitting part to the light receiving part using the light shielding wall.

In the embodiment, the light shielding member may further have a diaphragm part.

Thereby, it may be possible to suppress incidence of light causing noise to the light receiving part.

In the embodiment, the light shielding wall may be formed by sheet-metal processing, and the diaphragm part may be formed by the sheet-metal processing or injection molding.

Thereby, it may be possible to form the light shielding wall and the diaphragm part using the respective suitable techniques.

Another aspect of the invention relates to a photodetection unit including a light emitting part that outputs light to an object, a light receiving part that receives light from the object, a first substrate on which the light emitting part is mounted, and a second substrate on which the light receiving part is mounted, wherein, supposing that a height of the first substrate from a reference surface set at an opposite side to the object with respect to the first substrate is h4 and a height of the second substrate from the reference surface is h5, h5>h4 is satisfied.

In the another aspect of the invention, the height from the reference surface to the second substrate on which the light receiving part is mounted is larger than the height to the first substrate on which the light emitting part is mounted. Thereby, the light emitting part and the light receiving part may be separately provided on the first and second substrates, and it may be possible to suppress the difference in height between the light emitting part and the light receiving part to be smaller and efficiently place devices in the photodetection unit or an apparatus including the photodetection unit.

In the embodiment, the second substrate may shield direct light from the light emitting part to the light receiving part.

Thereby, it may be possible to use the second substrate as the light shielding member that shields direct light.

In the embodiment, in a state in which an apparatus including the photodetection unit is attached to a subject as the object, supposing that a distance from the first substrate to the object is d1 and a distance from the second substrate to the object is d2, d1>d2 may be satisfied.

Thereby, it may be possible to define relative position relationships among the respective parts of the photodetection unit using the distances from the first and second substrates to the object.

In the embodiment, supposing that a height of the light emitting part from the reference surface is h6 and a height of the light receiving part from the reference surface is h7, h7>h6 may be satisfied.

Thereby, it may be possible to define relative position relationships among the respective parts of the photodetection unit using the distances from a given reference surface to the light emitting part and the light receiving part.

In the embodiment, the light emitting part may have a lens part that condenses light, an enclosing part in which the light emitting device is enclosed, and a base part as a base of the enclosing part, and the height h6 of the light emitting part may be a height of the lens part from the reference surface.

Thereby, it may be possible to use the height of the lens part as the height of the light emitting part.

In the embodiment, a second light receiving part that outputs a detection signal for noise reduction is further provided, and the second light receiving part may be mounted on the first substrate.

Thereby, it may be possible to mount the light receiving part for noise reduction on the first substrate.

In the embodiment, a second light receiving part that outputs a detection signal for noise reduction is further provided, and the second light receiving part may be mounted on the second substrate.

Thereby, it may be possible to mount the light receiving part for noise reduction on the second substrate.

In the embodiment, supposing that a distance between the light emitting part and the light receiving part is L1 and a distance between the light emitting part and the second light receiving part is L2, L1<L2 may be satisfied.

Thereby, it may be possible to appropriately set the relationship of the two light receiving parts mounted on the second substrate in distance with the light receiving part.

In the embodiment, the first substrate and the second substrate may be integrally formed by a flexible board.

Thereby, it may be possible to integrally form the first and second substrates, not as different two substrates.

In the embodiment, supposing that a surface of the first substrate on which the light emitting part is mounted is a first surface, a rear surface of the first surface of the first substrate is a second surface, and a direction from the first surface to the second surface is a first direction, the reference surface may be a surface located at a side in the first direction of the second surface and in parallel to the second surface.

Thereby, it may be possible to set the reference surface based on the respective surfaces of the first substrate.

Further, the embodiment relates to a photodetection unit including a light emitting part that outputs light to an object, a light receiving part that receives light from the object, a first substrate on which the light emitting part is mounted, and a second substrate on which the light receiving part is mounted, wherein, in a state in which an apparatus including the photodetection unit is attached to a subject as the object, supposing that a distance from the first substrate to the object is d1 and a distance from the second substrate to the object is d2, d1>d2 may be satisfied.

In the other embodiment of the invention, the distance from the first substrate on which the light emitting part is mounted to the object is larger than the distance from the second substrate on which the light receiving part is mounted to the object. Thereby, the light emitting part and the light receiving part may be separately provided on the first and second substrates, and it may be possible to suppress the difference in height between the light emitting part and the light receiving part to be smaller and efficiently place devices in the photodetection unit or an apparatus including the photodetection unit.

Furthermore, another aspect of the invention relates to a biological information detection apparatus including the above described photodetection unit.

As below, the embodiments of the invention will be explained. The embodiments to be explained are not unduly limit the contents of the invention described in the appended claims. Further, not all of the configurations explained in the embodiments are necessarily essential component elements of the invention.

### 1. Technique of Embodiments

First, a technique of the embodiments will be explained. As described above, in a detection apparatus of biological information etc., it is necessary to detect high-quality signals in a photodetection unit. For the purpose, a high-performance light emitting part (e.g., an LED) is effectively used. Various points of view are conceivable for performance evaluation of the light emitting part and, here, higher brightness and smaller luminous flux angles are assumed. Such a light emitting part applies strong light to the more limited range and, for example, the intensity of the reflected light reflected by a living organism is also higher. As a result, it may be possible to make the intensity of light received by the light receiving part higher.

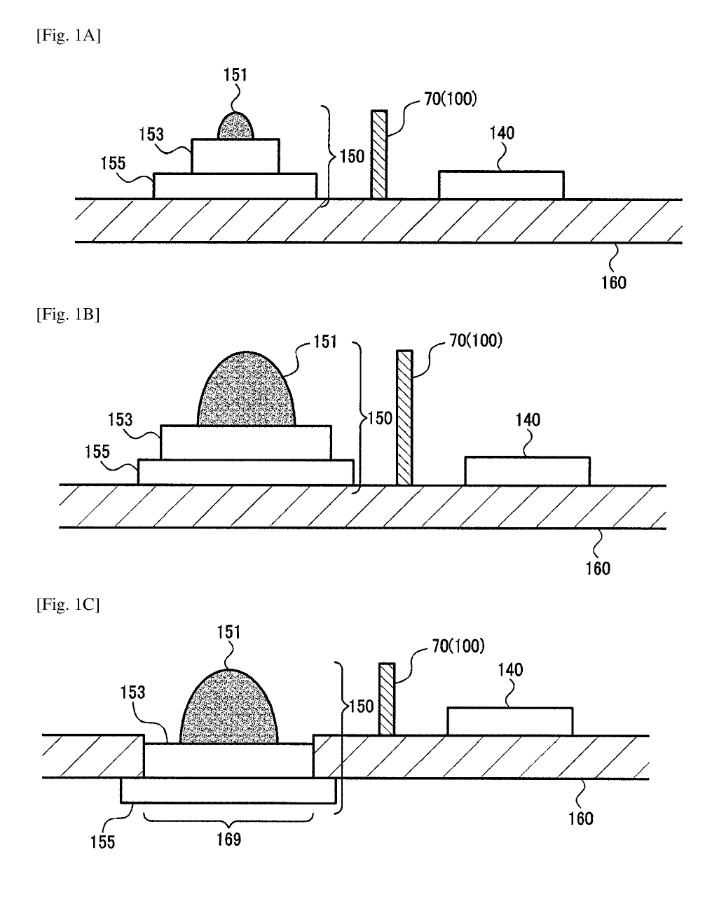

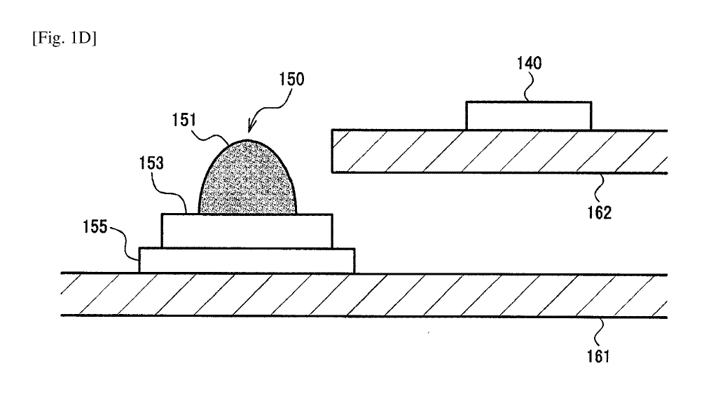

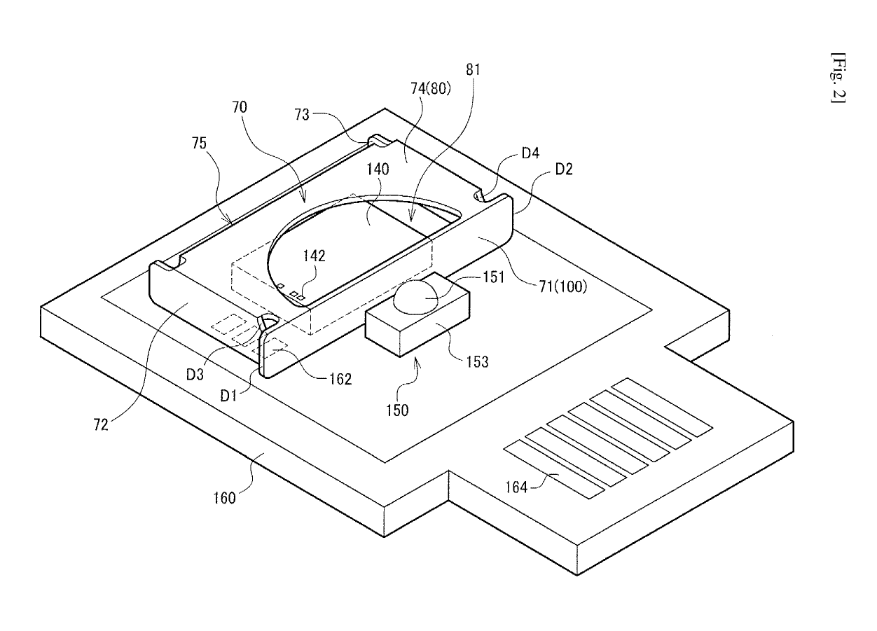

However, the size of the light emitting part is larger than that of a light emitting part having lower performance. Particularly, suppose that a direction perpendicular to the substrate surface is a height direction, the height is larger. FIGS. 1A and 1B show examples of photodetection units of comparative examples.

FIG. 1A is a sectional view of the photodetection unit of the first comparative example, and FIG. 1B is a sectional view of the photodetection unit of the second comparative example. In the second comparative example, a light emitting part with higher performance than that in the first comparative example is provided. As shown in FIGS. 1A and 1B, in either case, the photodetection unit includes a substrate 160, a light receiving part 140, a light emitting part 150, and a light shielding member 70. Note that, in FIG. 1A etc., a light shielding wall 100 of the light shielding member 70 is shown, however, the light shielding member 70 may include a diaphragm part 80 or the like. The light shielding member 70 shields direct light from the light emitting part 150 to the light receiving part 140 and disturbance light, and thereby, suppresses noise in detection signals.

Further, the light emitting part 150 of either FIG. 1A or 1B includes a lens 151, an enclosing part 153 in which a light emitting device is enclosed, and a base part 155 as a base of the enclosing part 153. Note that, as described above, the light emitting part 150 with the higher performance of FIG. 1B has the larger size, particularly, the larger height than that with the lower performance of FIG. 1A. Here, the height refers to a length in a direction intersecting with (in a strict sense, a direction orthogonal to) the substrate surface.

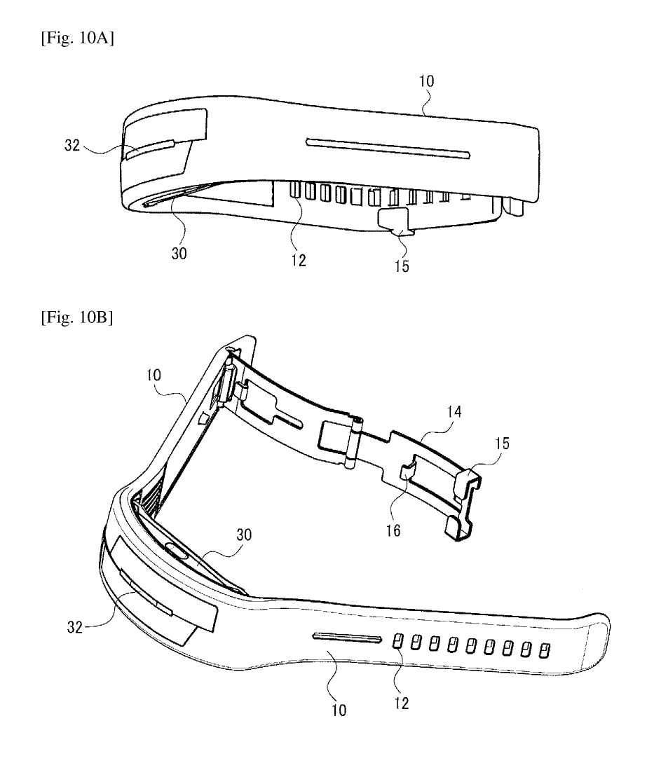

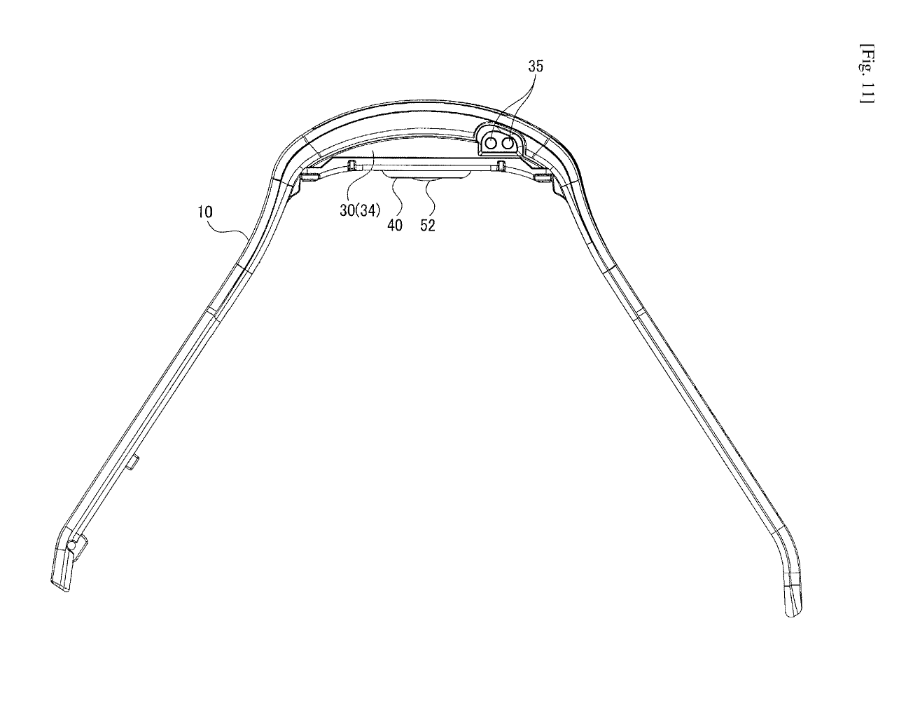

Accordingly, in the first comparative example shown in FIG. 1A, the height is larger than that in the example using the light emitting part without the lens part 151, and, in the light emitting part 150 of the second comparative example shown in FIG. 1B, the height is even larger. Resulting two disadvantages are roughly conceivable. First, the photodetection unit (and the apparatus including the photodetection unit) has a certain thickness and difficult to be downsized. As will be described later using FIG. 10A etc., a wearable apparatus of a wristwatch type or the like is assumed as a biological information detection apparatus including the photodetection unit. In this case, it is necessary that a user continuously wears the apparatus in a measurement period, difficulty in downsizing is a significant problem in a viewpoint that a feeling of discomfort may not be provided to the user or the user's action may not be hindered.

Second, the difference in height between the light emitting part 150 and the light receiving part 140 is larger. How much the photodetection unit is made closer to an object (an object to which light is applied, in a strict sense, a living organism) depends on the highest part of the photodetection unit. That is, when the height of the light emitting part 150 becomes larger and the difference from the height of the light receiving part 140 becomes too large, however close the photodetection unit is made to the object, it is impossible that the light receiving part 140 comes sufficiently closer to the light receiving part 140. As a result, the optical path to reception of the reflected light by the object in the light receiving part 140 becomes longer and the level of the detection signal in the light receiving part 140 becomes lower. Note that, in the embodiment having the light shielding member 70, in a strict sense, it is assumed that the highest part of the photodetection unit is the light shielding member 70, not the light emitting part 150. However, the height of the light shielding member 70 is set to be equal to or larger than the height of the light emitting part 150, and the problem of the height difference between the light emitting part 150 and the light receiving part 140 is unchanged.

Here, the problem of the height difference may be solved by making the light receiving part 140 higher by providing a part as the base of the light receiving part 140 or the like. However, even in this case, it is impossible to address the problem of difficulty in downsizing of the photodetection unit.

Accordingly, the applicant proposes a technique of mounting the light emitting part 150 on the substrate 160 as shown in FIG. 1C. Specifically, the photodetection unit includes a substrate 160, a light emitting part 150 that outputs light to an object, and a light receiving part 140 attached to the substrate 160 and receiving the light from the object, and a hole part 169 is provided in the substrate 160 and the light emitting part 150 is attached into the hole part 169 of the substrate 160.

According to the configuration, the height of the light emitting part 150 may be absorbed by the amount corresponding to the depth of the hole part. Therefore, downsizing may be realized by suppressing the thickness (height) of the photodetection unit itself, and, as is clear from the comparison between FIG. 1C and FIG. 1B, the difference in height between the light emitting part 150 and the light receiving part 140 may be reduced.

Further, in the embodiment, a form without the light shielding member 70 is conceivable. In the photodetection unit of the embodiment, it is assumed that the reflected light applied from the light emitting part 150 to the object (e.g., subject) and reflected by the object is detected by the light receiving part 140. That is, direct light from the light emitting part 150 to the light receiving part 140 contributes to noise, and, in the related art of PTL 3 etc. and the comparative examples 1 and 2 shown in FIGS. 1A and 1B, it has been necessary to provide the light shielding member 70 that shields at least the direct light.

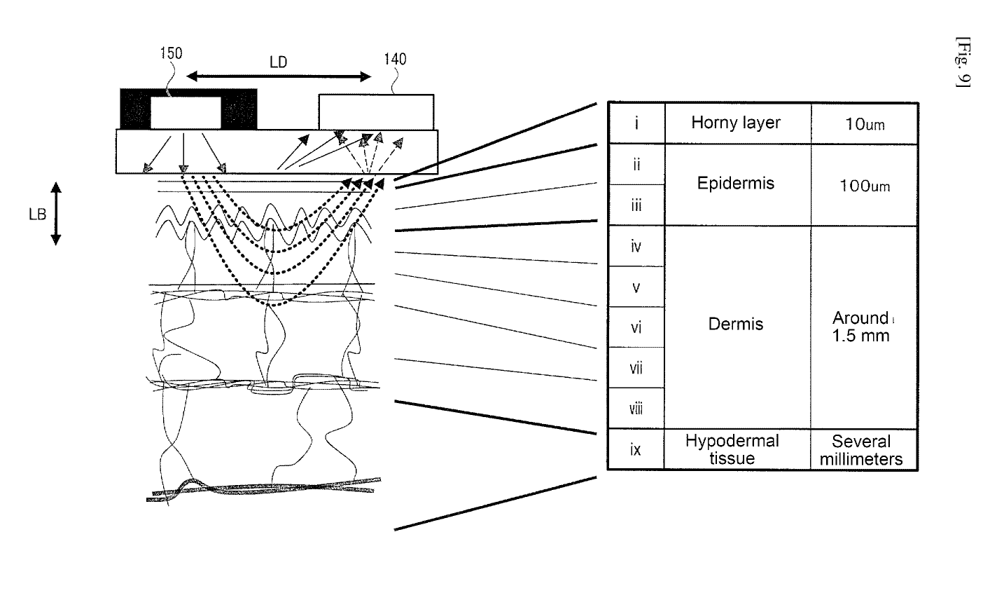

If the light shielding member 70 is essential, the number of parts is larger. Further, as will be described later using FIG. 8, it is preferable that the distance between the light emitting part 150 and the light receiving part 140 is smaller to a certain degree, however, there has been a risk that the distance is not sufficiently made smaller because of the existence of the light shielding member 70 (particularly, the light shielding wall 100).

Accordingly, the applicant proposes a technique of separately providing a substrate on which the light emitting part 150 is provided and a substrate on which the light receiving part 140 is provided. Specifically, as shown in FIG. 1D, the photodetection unit according to the embodiment includes a light emitting part 150 that outputs light to an object, a light receiving part 140 that receives the light from the object, a first substrate 161 on which the light emitting part 150 is mounted, and a second substrate 162 on which the light receiving part 140 is mounted. Further, supposing that a height of the first substrate from a reference surface set at the opposite side to the object with respect to the first substrate 161 is h4 and a height of the second substrate 162 from the reference surface is h5, h5>h4 holds.

Here, the reference surface is a surface as reference when the height is considered, and set at the opposite side (the downside in FIG. 1D) to the object side (in a direction in which the light is output, the upside in FIG. 1D) with respect to the first substrate 161. The reference surface here is a surface in parallel to the first substrate 161 in a strict sense. Or, when the apparatus in which the photodetection unit is incorporated (biological information detection apparatus) has a surface in contact with the object and a surface at the rear side (LCD side), the reference surface may be set to the rear side surface. When the biological information detection apparatus is as shown in FIGS. 10A and 10B, the reference surface may be set to a surface on which a light emitting window part 32 is provided or a plane determined based on the surface.

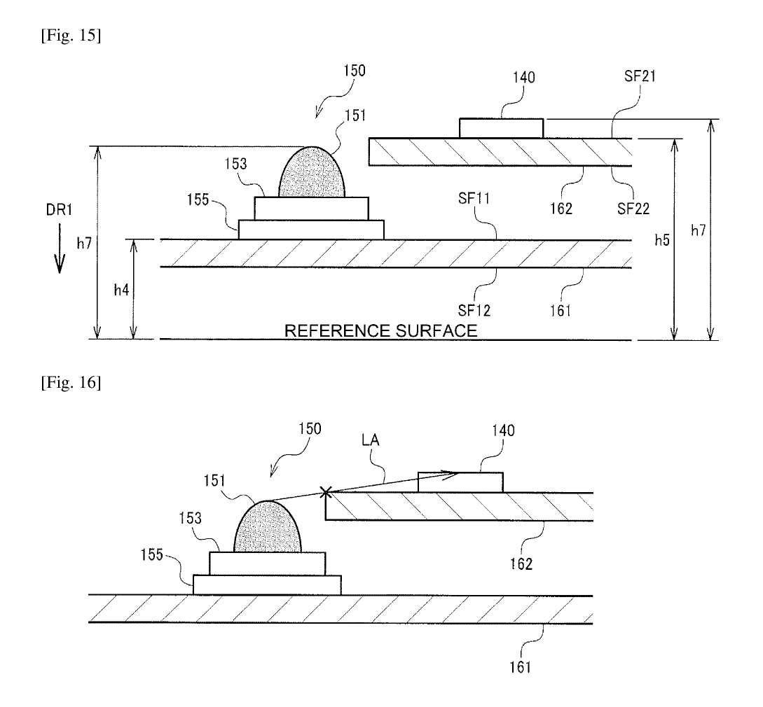

Or, supposing that the surface of the first substrate 161 on which the light emitting part 150 is mounted is a first surface (SF11 in FIG. 15), the rear surface of the first surface SF11 of the first substrate 161 is a second surface (SF12 in FIG. 15), and a direction from the first surface SF11 to the second surface SF12 is a first surface (DR1 in FIG. 15), the reference surface may be a surface located at the side in the first direction DR1 of the second surface SF12 and in parallel to the second surface SF12.

In this manner, the reference surface may be set based on the respective surfaces of the first substrate 161. The reference surface in this case is the surface shown in FIG. 15. Note that it is not necessary to provide some member in the location of the reference surface, but the reference surface may be a hypothetical surface.

Further, the explanatory diagram of h4, h5 is FIG. 15 and when the reference surface as shown in FIG. 15 is set, h4 and h5 correspond to the distances respectively shown. FIG. 15 shows an example of using distances to the surface at the object side (the side farther from the reference surface) of the two surfaces of the respective substrates as the heights to the first substrate 161 and the second substrate 162. The surface at the object side is the above described first surface SF11 in the first substrate 161, the surface shown by SF21 in FIG. 15 in the second substrate 162, and, in a strict sense, the surface on which the light receiving part 140 is mounted.

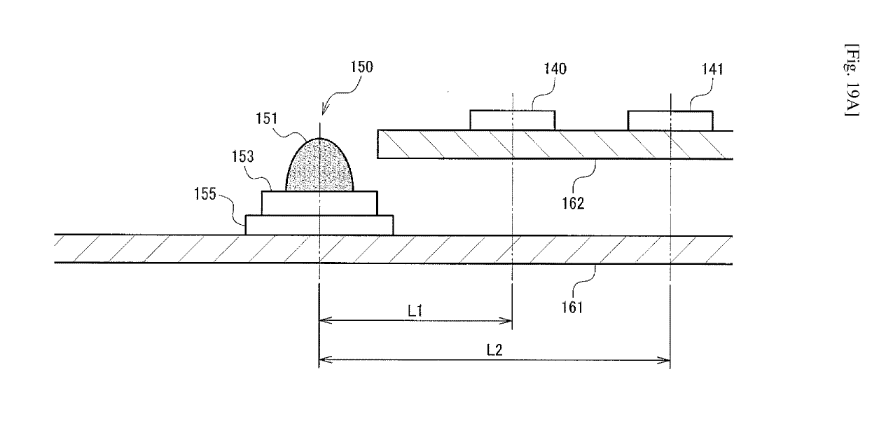

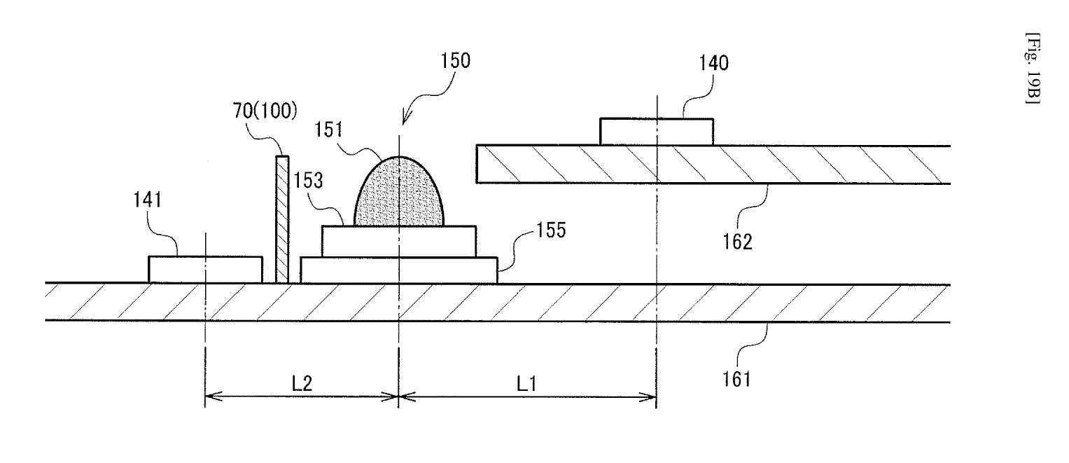

According to the configuration, the heights from the substrate surfaces are different between the first substrate 161 and the second substrate 162 in the first place. Therefore, if h5>h4, i.e., the first substrate 161 at the light emitting part 150 side is set lower than the second substrate 162 at the light receiving part 140 side, the difference in height between the light emitting part 150 and the light receiving part 140 may be absorbed by the height difference. In this case, a plurality of substrates are provided in the height direction (or, as will be described later using FIG. 18, one substrate is multiply folded), and the effect in downsizing of the photodetection unit itself is not extremely significant. However, other devices may be placed on the first substrate 161 and the second substrate 162, and thus, in the case of consideration as the photodetection unit when the other devices are added or the biological information detection apparatus including the photodetection unit, efficient device arrangement may be possible and downsizing as a whole may be realized.

Note that it is impossible to consider the relative relationship between the height h4 of the first substrate 161 and the height h5 of the second substrate 162 from the reference surface unless the position relationship between the first substrate 161 and the second substrate 162 is determined. h4 and h5 in the embodiments may be set to the heights from the reference surface in the state in which the first substrate 161 and the second substrate 162 (and light emitting part 150 and the light receiving part 140) are mounted as the photodetection unit. Specifically, when the photodetection unit according to the embodiments is incorporated in the biological information detection apparatus as shown in FIGS. 10A to 11, h4 and h5 show the heights from the reference surface in a state in which the first substrate 161 and the second substrate 162 are incorporated in the biological information detection apparatus and fixed.

In this regard, as shown in FIG. 16, the position relationship may be set so that the second substrate 162 may shield direct light (LA in FIG. 16) from the light emitting part 150 to the light receiving part 140.

In this manner, it may be possible to shield direct light using the second substrate 162 itself without providing the light shielding wall 100 as shown in FIGS. 1A and 1B. Accordingly, reduction in the number of parts, reduction in the distance between the light emitting part 150 and the light receiving part 140 along the substrate surface, etc. may be realized.

Note that, in the photodetection unit of the embodiments, not only direct light but also light entering from outside and reflected light reflected by others than the object may cause noise. Therefore, even in the case without the light shielding wall 100, provision of the light shielding member 70 that shields the disturbance light is not hindered.

As below, the embodiments will be explained in detail. Specifically, the configuration example of the photodetection unit corresponding to FIG. 1C will be explained as the first embodiment, and the configuration example of the photodetection unit corresponding to FIG. 1D will be explained as the second embodiment. Finally, a specific example of the biological information detection apparatus including the photodetection unit will be explained.

### 2. First Embodiment

2.1 Photodetection Unit

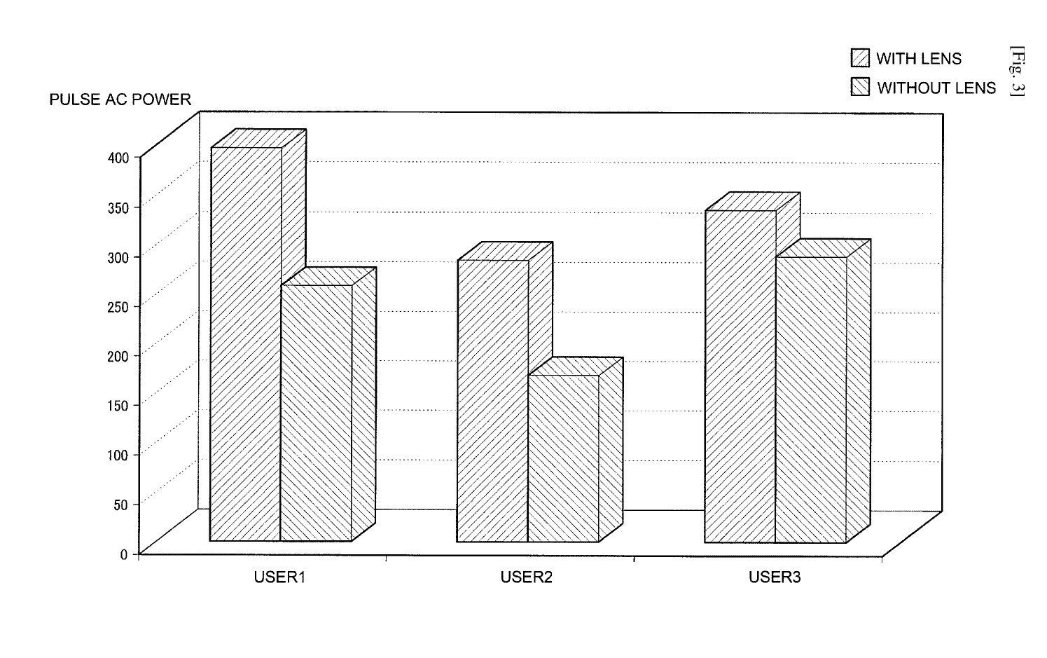

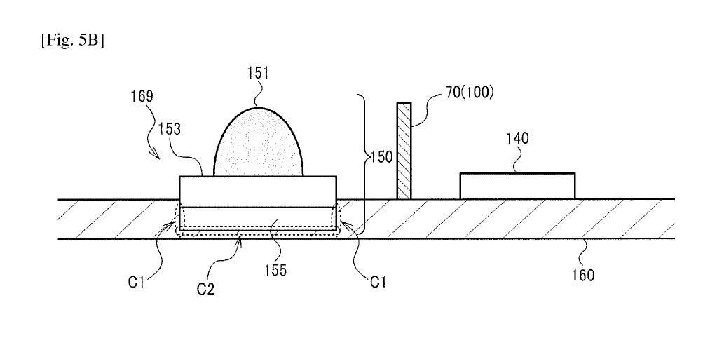

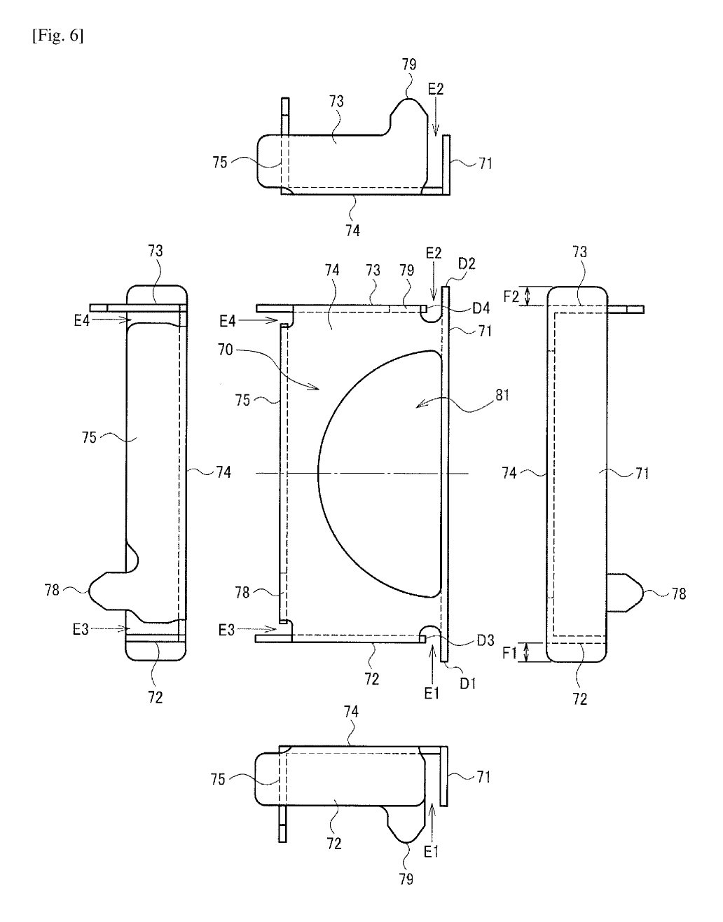

FIG. 2 is a perspective view showing a configuration example of the photodetection unit of the embodiment. As described above, the photodetection unit of the embodiment includes the substrate 160, the light receiving part 140, the light emitting part 150, and the light shielding member 70. FIG. 2 shows the example in which the enclosing part 153 of the light emitting part 150 projects to the first surface (the surface on which the light receiving part 140 is mounted) side. This corresponds to e.g., the case where the thickness (height) of the enclosing part 153 is larger than that of the substrate 160 and, as will be described later using FIG. 5B, the case where the sum of the thicknesses of the enclosing part 153 and the base part 155 is larger than the depth of the hole part 169 or the like. Note that, as shown in FIG. 1C, the enclosing part 153 not projecting to the first surface side may be employed.

The light emitting part 150 outputs light to an object (subject or the like) and the light receiving part 140 receives the light from the object. For example, when the light emitting part 150 outputs light and the light is reflected by the object, the light receiving part 140 receives the reflected light. The light receiving part 140 may be realized using a light receiving device such as e.g. a photodiode. The light emitting part 150 may be realized using a light emitting device such as e.g. an LED. For example, the light receiving part 140 may be realized using a P-N junction diode device formed on a semiconductor substrate or the like. In this case, an angle-limiting filter that narrows down the light receiving angle and a wavelength-limiting filter that limits the wavelength of light entering the light receiving device may be formed on the diode device.

In the case where the unit is applied to a biological information detection apparatus such as a pulsimeter as an example, the light from the light emitting part 150 travels inside of the subject as the object and disperses or scatters in epidermal, dermal, hypodermal tissues or the like. Then, the light reaches a vessel (part to be detected) and is reflected. In this regard, part of the light is absorbed by the vessel. Then, the absorptance of the light in the vessel changes due to pulse and the amount of reflected light changes. The light receiving part 140 receives the reflected light and detects the changes of the amount of light, and thereby, the pulse rate etc. as biological information may be detected.

Note that the dome lens 151 (a condenser lens in a broad sense, also referred to as “lens part”) provided in the light emitting part 150 is a lens for condensing light from an LED chip (a light emitting device chip in a broad sense, also referred to as “light emitting device”) resin-sealed (sealed with a light-transmissive resin) in the light emitting part 150. That is, in the surface-mounted light emitting part 150, the LED chip is provided below the dome lens 151 and the light from the LED chip is condensed by the dome lens 151 and output to the object. Thereby, the optical efficiency of the photodetection unit may be improved.

FIG. 3 shows differences in detection signal level (here, AC power of pulse signal) of the photodetection unit with or without the lens part 151. Here, signal levels in the cases with the lens part 151 and without the lens part 151 were measured with respect to the three different users. As is clear from FIG. 3, though the degrees of improvement in the signal level are different, it was confirmed that the signal level is improved with the lens part 151 for all users.

The sectional view of the photodetection unit is as shown in FIG. 1C, and the light receiving part 140 is attached to the first surface of the substrate 160 and the light emitting part 150 is inserted into the hole part 169 from the second surface side of the substrate as the rear surface (opposite surface) of the first surface.

Here, the substrate 160 has a thickness, and, if the substrate is a flat plate-like substrate, the substrate has a parallelepiped shape having six surfaces in a strict sense. However, the surface in the height direction (the surface shown as the substrate 160 in FIG. 1C) has a very small area and arrangement of devices thereon is not assumed. That is, supposing that the flat substrate as the parallelepiped has a size of width W*depth D*height H (here, W, D>>H), the first and the second surfaces are surfaces having areas of W*D. Further, a substrate not a flat plate like a flexible substrate may be employed, and, in this case, the first and second surfaces may be considered as two surfaces having larger areas on which arrangement of devices is assumed.

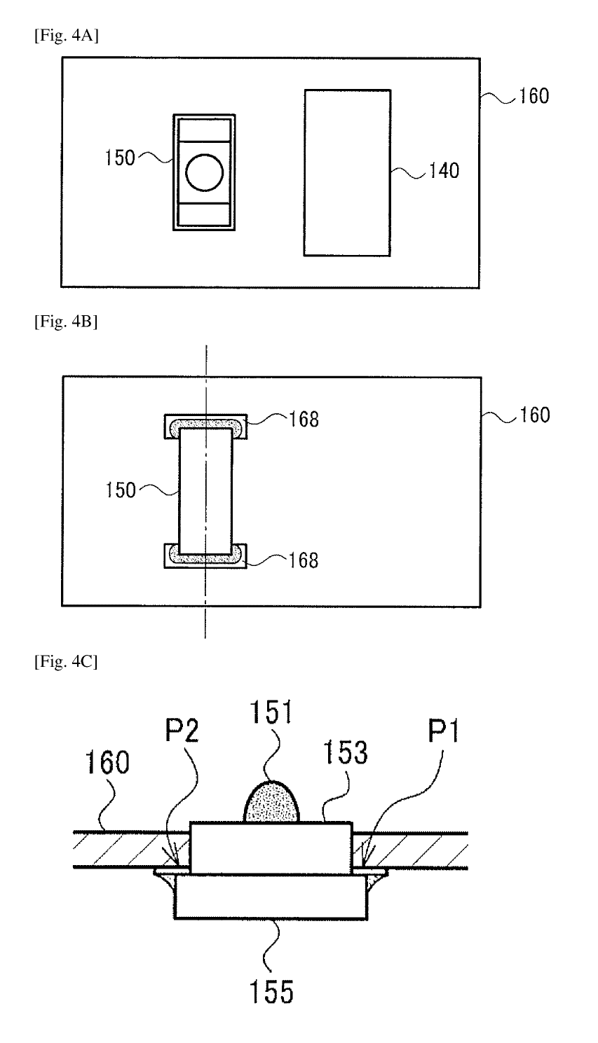

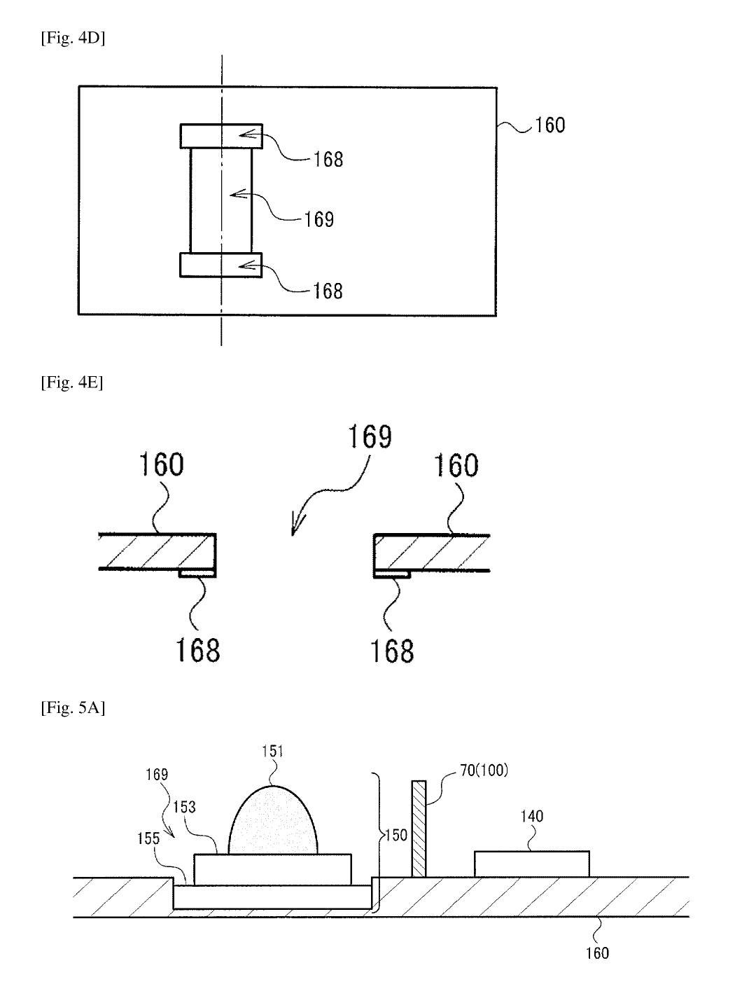

As described above, the first surface here is the surface on which the light receiving part 140 is provided of the substrate 160 and the upper surface in FIG. 1C. FIG. 4A shows a position relationship between the light receiving part 140 and the light emitting part 150 on the first surface. In this case, the second surface is the lower surface in FIG. 1C and the light emitting part 150 is attached from the lower side to the upper side of the hole part 169 as shown in FIG. 1C. In this manner, the hole part 169 having the depth for the thickness of the substrate is used, and it may be possible to absorb the height of the light emitting part 150 by the amount of the thickness of the substrate.

FIG. 4B shows an arrangement example of the light emitting part 150 on the second surface. As described above, the light receiving part 140 is provided on the first surface and not visually recognized from the second surface side. In the example of FIG. 4B, lands 168 for connection are provided on the substrate 160 and the light emitting part 150 is fixed to the lands 168 by soldering.

Further, the light emitting part 150 has the lens part 151 that condenses light (the above described dome lens in a strict sense), and the light emitting part 150 may be inserted into the hole part 169 so that the lens part 151 may project to the first surface side.

For example, in FIG. 1C, the most part of the lens part 151 projects to the upper surface side, i.e., the first surface side of the substrate 160. As described above, suppression of the height of the light emitting part 150 is important in consideration of downsizing, however, if the height is made to be excessively smaller, the difference in height between the light emitting part 150 and the light receiving part 140 is problematic. For example, when the end of the lens part 151 does not project higher to the first surface side than the hole part 169, in contrast to the above described example, a problem that it is impossible to make the optical path length shorter because the light emitting part 150 is lower than the light receiving part 140 may arise. Further, in consideration of the thickness of the substrate 160 and the size of the light emitting part 150 under the current situation, it is natural that the lens part 151 is mounted through the substrate 160. That is, when the light emitting part is inserted into the hole part 169 from the second surface side, the lens part 151 projects to the first surface side.

The mounting technique of the light emitting part 150 of the embodiment is not limited to a technique of providing the hole part 169 through the substrate and inserting the light emitting part 150 from the second surface side to project to the first surface side. For example, as shown in FIG. 5A, a non-through hole part 169 may be provided at the first surface side of the substrate 160 and the light emitting part 150 may be inserted into the hole part 169 from the first surface side. In this case, the height that can be absorbed is less than that in FIG. 1C, however, there is an advantage that the height of the light emitting part 150 is suppressed without projection of the base part 155 to the rear surface.

Further, as shown in FIG. 5B, the widths of the base part 155 and the enclosing part 153 in the section view may be the same. According to the configuration, the size of the hole part 169 formed in the substrate 160 may be restricted to the minimum necessary.

In the configuration in FIG. 5A or 5B, in the light emitting part 150, the terminals electrically connected to the light emitting device may be provided on the bottom surface or the side surfaces of the base part 155. The lands 168 at the substrate 160 side are provided in the locations corresponding to the terminals of the light emitting device, i.e., in the bottom surface part or the side surface part of the hole part 169. Specifically, when the terminals and the lands 168 are provided on the side surfaces, they are connected in the regions shown by C1 in FIG. 5B and, when the terminals and the lands 168 are provided on the bottom surface, they are connected in the region shown by C2 in FIG. 5B. According to the configuration, an advantage that the height of the light emitting part 150 is suppressed without projection of the base part 155 to the rear surface may be obtained.

As shown in FIG. 1C, the light emitting part 150 may have the light emitting device (not shown), the enclosing part 153 in which the light emitting device is enclosed, and the base part 155 as the base of the enclosing part 153. Further, the base part 155 is provided at the second surface side in a state in which the light emitting part 150 is inserted into the hole part 169. Particularly, the base part 155 may have terminals electrically connected to the light emitting device and electrically connected to wires provided on the second surface of the substrate 160.

A specific example will be explained using the drawings. The enclosing part 153 is a part in which the light emitting device is enclosed, and the lens part 151 is located closer to the object side than the enclosing part. Further, the base part 155 serves as the base of the enclosing part 153 and is located at the side in a different direction from the object. That is, in the light emitting part 150, as shown in FIG. 1C etc., the lens part 151, the enclosing part 153, and the base part 155 are sequentially provided along the direction from the object side toward the substrate 160 and stability etc. when the device is mounted is considered, and thereby, the respective section areas generally satisfy lens part 151<enclosing part 153<base part 155 as shown in FIG. 1C.

In the case where the light emitting part 150 is inserted into the hole part 169 from the second surface side, insertion of the whole including the base part 155 into the hole part 169 is not hindered, however, as shown in FIG. 1C, the base part 155 may be left at the second surface side. In this manner, fixation of the light emitting part 150 to the substrate 160 is easier and the part projecting to the first surface side of the light emitting part 150 is made smaller, and thereby, the difference in height between the light emitting part 150 and the light receiving part 140 may be reduced.

In the light emitting part 150, the terminals electrically connected to the light emitting device are generally provided in the base part 155, and, for example, the terminals are provided in locations shown by P1, P2 in FIG. 4C of the base part 155. In the case where the terminals are provided in P1, P2, the light emitting part 150 is inserted in the state of FIG. 1C, P1, P2 are the locations in contact with the second surface of the substrate 160 as shown in FIG. 4C. That is, regarding the substrate 160, the wires are provided in appropriate locations at the second surface side, and thereby, connection of the light emitting part 150 to the terminals is easier.

For example, FIG. 4D shows the substrate 160 in which the light emitting part 150 is not inserted as seen from the second surface side, and the lands 168 for connection are provided around the hole part 169 as shown in FIG. 4D. Further FIG. 4E is a sectional view of the substrate 160 in which the light emitting part 150 is not inserted. In the arrangement, the light emitting part 150 is inserted into the hole part 169 as shown in FIG. 4C, and thereby, P1, P2 in which the terminals are provided and the lands 168 appropriately respond. Accordingly, it may be possible to easily mount the light emitting part 150 on the substrate 160 by soldering as shown in FIG. 4C.

The light shielding member 70 is a member that shields light. For example, in FIG. 2, the light shielding member 70 shields the light receiving part 140 from light. That is, the light shielding member 70 is not provided at the light emitting part 150 side, but provided at the light receiving part 140 side. For example, the light shielding member 70 is provided to cover the light receiving part 140 to shield incident light to the light receiving part 140, but not to shield the light emitting part 150 from light. Note that a modification in which the light shielding member 70 is provided at the light emitting part 150 side may be made.

It is desirable to perform reflection suppression processing on at least the inner surface of the light shielding member 70. For example, the color of the surface (inner side surface etc.) of the light shielding member 70 is made to be a predetermined color such as black so that diffused reflection of light may be prevented. Or, the surface of the light shielding member 70 may be formed in a moth-eye structure. For example, a concavo-convex structure with a period of several tends to several hundreds of nanometers is formed and used as an anti-reflection structure. By the reflection suppression processing, for example, a situation that stray light of the reflected light on the surface of the light shielding member 70 turns to the noise component of the detection signal may be effectively suppressed.

The light receiving part 140, the light emitting part 150, and the light shielding member 70 are mounted on the substrate 160. The substrate 160 is e.g., a rigid substrate. On the substrate 160, terminals 162 for connecting terminals 142 of signals and power of the light receiving part 140 and terminals 164 for connecting signals and power between an external main substrate and the substrate are provided. For example, the terminals 142 of the light receiving part 140 and the terminals 162 of the substrate 160 are connected by wire bonding or the like.

In the embodiment, the light shielding member 70 is formed by sheet-metal processing of a metal (e.g., an alloy of tin and copper). For example, the light shielding member 70 having the shape as shown in FIG. 2 is formed by sheet-metal processing of one metal plate. Further, the light shielding member 70 has the light shielding wall 100 provided between the light emitting part 150 and the light receiving part 140. The light shielding wall 100 shields the light from the light emitting part 150 (direct light or the like) entering the light receiving part 140. Furthermore, the light shielding wall 100 is formed by a first metal surface 71 of the light shielding member 70 formed by sheet-metal processing. That is, the first metal surface 71 as the light shielding wall 100 is provided between the light receiving part 140 and the light emitting part 150, and thereby, the light from the light emitting part 150 entering the light receiving part 140 is suppressed.

In addition, the light shielding member 70 has second and third metal surfaces 72 and 73. These second and third metal surfaces 72 and 73 are provided along a direction intersecting with (e.g., orthogonal to) the first metal surface 71. For example, assuming that the first metal surface 71 is the metal surface at the front side, the second and third metal surfaces 72 and 73 are the metal surfaces at the side surface sides to form the light shielding walls at the side surface sides.

As shown in FIG. 2, a first end surface (left end surface) shown by D1 of the first metal surface 71 projects toward one side (left side) more than an end surface shown by D3 of the second metal surface 72 in the front view of the first metal surface 71 as seen from the light emitting part 150 side. On the other hand, a second end surface (right end surface) shown by D2 opposed to the first end surface of the first metal surface 71 projects to the other side (right side) different from the one side more than an end surface shown by D4 of the third metal surface 73 in the front view. That is, the end surfaces shown by D1 and D2 of the first metal surface 71 project to both sides more than the end surfaces shown by D3 and D4 of the second and third metal surfaces.

For example, the first metal surface 71 and the second metal surface 72 are adjacently provided via a first gap region shown by E1 in FIG. 6, which will be described later. Further, the first metal surface 71 and the third metal surface 73 are adjacently provided via a second gap region. That is, the rear surface of the first metal surface 71 and the end surfaces shown by D3 and D4 of the second and third metal surfaces are not in contact and the gap regions exist between the rear surface and the end surfaces.

If the gap regions exist, as will be described later in detail, there is a risk that the light from the light emitting part 150 enters the light receiving part 140 via the gap regions. However, in the embodiment, as described above, the end surfaces shown by D1 and D2 of the first metal surface 71 project to both sides more than the second and third metal surfaces 72 and 73 in the front view, and thus, the situation that the light from the light emitting part 150 enters the light receiving part 140 may be effectively suppressed.

Further, the light shielding member 70 has a fourth metal surface 74 provided along a direction intersecting with (e.g., orthogonal to) the first metal surface 71 and shielding incidence of the light to the light receiving part 140. The fourth metal surface 74 is e.g., a metal surface of the upper surface of the light shielding member 70.

Furthermore, the diaphragm part 80 that narrows down the light (reflected light or the like) from the object in the optical path between the object and the light receiving part 140 is formed in the fourth metal surface 74. That is, an opening part 81 of the diaphragm part 80 is formed in the fourth metal surface 74. Note that a fifth metal surface 75 as a light shielding wall of the rear surface is also provided in the light shielding member 70, and shields the light entering from the rear side surface.

2.2 Light Shielding Member

As shown in FIGS. 1C and 2, the light shielding member 70 that shields at least the light receiving part 140 is provided on the substrate 160. As below, the light shielding member 70 will be explained.

In the light detection unit of the embodiment, as shown in FIG. 2, the light shielding member 70 for shielding the light receiving part 140 etc. from external light is provided. Further, the light shielding member 70 is formed by sheet-metal processing of a metal, and the light shielding wall 100 is realized by e.g., the metal surface 71 of the light shielding member 70. The diaphragm part 80 having the opening part 81 is realized by e.g., the metal surface 74 of the light shielding member 70. Here, for example, the light shielding wall 100 has a wall surface along a direction intersecting with (orthogonal to) the line segment connecting the center position of the light receiving part 140 and the center position of the light emitting part 150. The light shielding wall 100 is provided, and thereby, incidence of the light (direct light) from the light emitting part 150 to the light receiving part 140 is suppressed and reliability of detection data or the like may be improved.

As will be described later in detail, as the distance between the light emitting part 150 and the light receiving part 140 is shorter, the optical efficiency and performance of the photodetection unit are improved. For example, the optical efficiency and performance are lower in inverse proportion to square of the distance. Therefore, it is desirable to minimize the distance between the light emitting part 150 and the light receiving part 140.

On the other hand, when the distance between the light emitting part 150 and the light receiving part 140 is made shorter, direct light from the light emitting part 150 enters the light receiving part 140 and increase in DC component or the like is caused and the performance is lower. On this account, in the photodetection unit of the embodiment, the light shielding wall 100 is provided between the light emitting part 150 and the light receiving part 140.

In this case, as a technique of a comparative example of the embodiment, a technique of forming the light shielding wall 100 of the light shielding member 70 by injection molding is conceivable. The technique of the comparative example using injection molding is advantageous in view of mass production of apparatuses or the like.

However, when the light shielding wall 100 is formed by injection molding, the wall thickness of the light shielding wall 100 is larger. That is, if the wall thickness of the light shielding wall 100 is designed to be smaller, the part of the light shielding wall 100 is not sufficiently filled with the resin at injection molding, and it is impossible to realize the light shielding wall 100 having a sufficient strength. Accordingly, in the technique of the comparative example using injection molding, the thickness of the light shielding wall 100 is e.g., 0.4 mm or more.

When the light shielding wall 100 is thicker, the distance between the light emitting part 150 and the light receiving part 140 is longer. Therefore, for example, the optical path length between the light emitting part 150 and the light receiving part 140 via the object is longer, and the optical efficiency and performance of the photodetection unit are lower.

Accordingly, in the embodiment, the light shielding member 70 is formed by sheet-metal processing of the metal. For example, FIG. 6 shows detailed shapes of the light shielding member 70 in a plan view, a side view, a front view, and a rear view. For example, one metal plate is bent by sheet-metal processing, and thereby, the light shielding member 70 including the metal surfaces 71, 72, 73, 74, 75 is formed. Specifically, the metal surfaces 71, 72, 73, 75 are bent at right angles (nearly right angles) with respect to the metal surface 74 as the upper surface, and thereby, the light shielding member 70 is formed.

The metal surface 71 opposed to the light emitting part 150 in FIG. 2 serves as the light shielding wall 100 that shields incidence of the direct light from the light emitting part 150 to the light receiving part 140. Further, in the metal surface 74 as the upper surface, the diaphragm part 80 that narrows down the light from the object in the optical path between the object and the light receiving part 140 is formed. That is, the diaphragm part 80 having the opening part 81 is formed.

As described above, when the light shielding wall 100 is realized using the metal surface 71 by sheet-metal processing, the thickness of the light shielding wall 100 may be made smaller compared to that by the technique of the comparative example using injection molding. For example, in the case of using sheet-metal processing, even when the thickness of the metal surface is e.g., about 0.1 mm, the light shielding member 70 having the sufficient strength may be realized. Accordingly, it may be possible to set the thickness of the metal surface 71 as the light shielding wall 100 to e.g., about 0.1 mm. Therefore, compared to the technique of the comparative example using injection molding by which the thickness of the light shielding wall 100 is e.g., 0.4 mm or more, the thickness of the light shielding wall 100 may be made sufficiently smaller, and the distance between the light emitting part 150 and the light receiving part 140 may be made shorter by the amount. Thus, it may be possible to shorten the optical path length of the light from the light emitting part 150 to the light receiving part 140 via the object while suppressing incidence of the direct light from the light emitting part 150 to the light receiving part 140 by the light shielding wall 100, and the detection performance of the photodetection unit or the like may be improved.

Particularly, in FIG. 2, the chip-package light emitting part 150 is used. In the chip-package light emitting part 150, for example, the dome lens 151 is provided on the LED chip, and thereby, emission efficiency of the light to the object is higher and the detection sensitivity of the photodetection unit may be improved.

However, the chip-package light emitting part 150 is larger in footprint than that of a type realized by providing an LED chip on a reflector, for example. Therefore, there is a problem that the distance between the light emitting part 150 and the light receiving part 140 is longer by the amount. In this regard, according to the embodiment, the thickness of the light shielding wall 100 is made sufficiently smaller as described above, and therefore, even when the chip-package light emitting part 150 is used, the case may be addressed and the detection performance of sensitivity of the photodetection unit or the like may be improved.

Further, in FIG. 2, the light shielding member 70 is not provided at the light emitting part 150 side, but only at the light receiving part 140 side. That is, the light shielding member 70 covers the light receiving part 140 for shielding light, but does not cover the light emitting part 150.

For example, if the light shielding member 70 is formed in a shape to shield also the light emitting part 150 from light, part of the light from the light emitting part 150 toward the object is shielded by the light shielding member 70, and there is a risk that the amount of light applied to the object or the like decreases and the detection performance of sensitivity or the like is lower.

In this regard, as shown in FIG. 2, the shape of the light shielding member 70 is formed to shield only the light receiving part 140 side from light, and thereby, occurrence of the situation that the output light from the light emitting part 150 is shielded by the light shielding member 70 and the amount of light to the object decreases may be suppressed.

Furthermore, the configuration in which the light shielding member 70 is not provided at the light emitting part 150 side, but only at the light receiving part 140 side is also advantageous in the viewpoint of reduction in thickness of the photodetection unit. As described above, the light emitting part 150 having the dome lens 151 (particularly, the light emitting part 150 with higher luminance and narrower luminous flux angle) has the larger height than the light receiving part 140. Even when the light emitting part is mounted in the hole part 169 shown in FIG. 1C etc., the height of the light emitting part 150 is generally larger than that of the light receiving part 140. Therefore, when the light shielding member 70 is provided at the light emitting part 150 side, the height at the light emitting part 150 side is larger by the amount and reduction in thickness of the photodetection unit is hindered.

In this regard, in the configuration in which the light shielding member 70 is provided at the light receiving part 140 side only, the light shielding member 70 does not exist at the light emitting part 150 side, and it may be possible to equalize the height at the light receiving part 140 side and the height at the light emitting part 150 side, for example. Therefore, compared to the technique of providing the light shielding member 70 also at the light emitting part 150 side, it may be possible to reduce the height of the photodetection unit as a whole and realization of reduction in thickness of the photodetection unit is easier.

As described above, the diaphragm part 80 is provided in the light shielding member 70. That is, the opening part 81 is formed in the metal surface 74 as the upper surface of the light shielding member 70, and the diaphragm part 80 is realized by the opening part 81. In this case, the opening part 81 of the diaphragm part 80 opens wider as being closer to the light emitting part 150. For example, the opening part 81 has a semi-circular shape (nearly semi-circular shape) and the diameter of the semi-circle is located at the light emitting part 150 side. The opening part 81 of the diaphragm part 80 is formed in the shape, and thereby, it may be possible to allow the light output from the light emitting part 150 and reflected by the object to efficiently enter the light receiving part 140, and the detection performance of sensitivity or the like may be improved. The details of the diaphragm part 80 will be described later.

A relationship between the height of the light shielding wall 100 of the light shielding member 70 and the heights of the light receiving part 140 and the light emitting part 150 will be explained. As described above, the light shielding member 70 has the light shielding wall 100 that shields the light from the light emitting part 150 entering the light receiving part 140. Supposing that the height of the light shielding wall 100 is h1 and the height of the light emitting part 150 is h2, h1>=h2 holds.

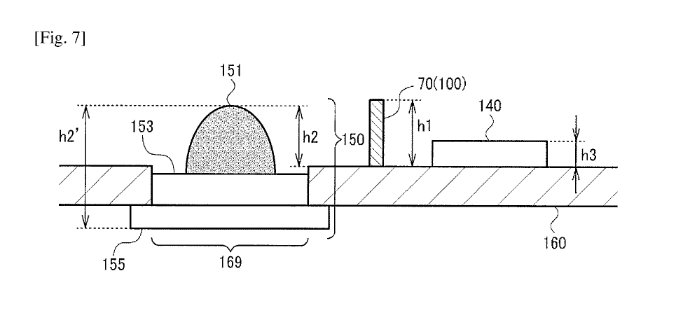

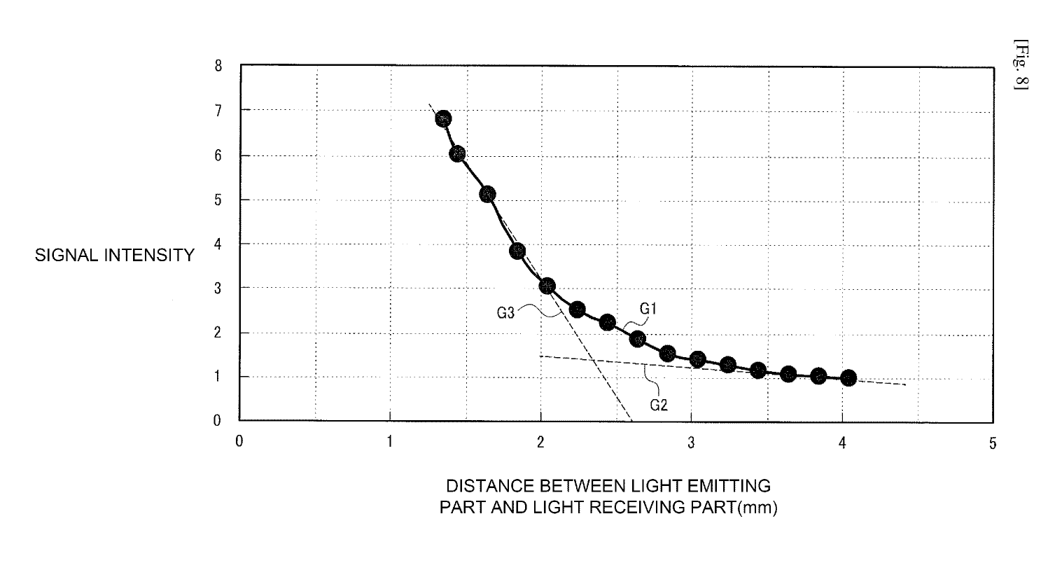

The heights here refer to the lengths in the height direction (the direction intersecting with, in a strict sense, the direction orthogonal to the substrate 160) from reference set to a given point. For example, in the case where the reference is set to the first surface of the substrate 160, the height h1 of the light shielding wall 100 and the height h2 of the light emitting part 150 are the heights shown in FIG. 7. That is, the height here does not represent the thickness of the light emitting part 150 itself (h2′ in FIG. 7).

The light shielding wall 100 here shields the direct light from the light emitting part 150 to the light receiving part 140. The minimum height of the light shielding wall 100 necessary for shielding the direct light depends on the height of the light receiving part 140, the distance between the light emitting part 150 and the light receiving part 140, etc., and shielding of the direct light is possible if the height is set to at least the height of the light emitting part 150 or more. Accordingly, here, the height relationship is set to h1>=h2. In this regard, supposing that the height of the light receiving part 140 is h3, h1>=h2>h3 may be set. The condition is realized using the light emitting part 150 and the light receiving part 140 having typical sizes, however, as described above, if the height difference is solved by providing a base in the light emitting part 150 or the like, the condition is not necessarily ensured. However, if h2<=h3, depending on the arrangement positions and the angles, there is a risk that the direct light is not shielded even when the requirement of h1>=h2 is satisfied. In this regard, if h1>=h2>3 is set, it may be possible to easily satisfy the condition for easily shielding the direct light.

In the above explanation, regarding the light shielding member 70, the light shielding wall 100 and the other parts (e.g., the diaphragm part 80) are formed by sheet-metal processing, however, not limited to that. For example, the light shielding wall 100 may be formed by sheet-metal processing and the diaphragm part 80 may be formed by sheet-metal processing or injection molding.

As will be described later, it is desirable to set the distance between the light emitting part 150 and the light receiving part 140 in a predetermined range. Accordingly, to flexibly set the distance, it is necessary to make the thickness of the light shielding wall 100 provided between the light emitting part 150 and the light receiving part 140 smaller. It may be possible that the above described sheet-metal processing forms a member to be thinner, and the sheet-metal processing is preferably used for the light shielding wall 100.

However, of the light shielding member 70, regarding the diaphragm part 80 provided above the light receiving part 140, some increase in thickness is not problematic. This is because the increase in thickness leads to increase in the height of the apparatus, however, the height of the light emitting part 150 is generally larger than the height of the light receiving part 140 as described above. Accordingly, even when the diaphragm part 80 provided at the light receiving part 140 side is thicker, the size of the photodetection unit is supposed to be determined based on the light emitting part 150. In consideration of the fact, the diaphragm part 80 may be formed by sheet-metal processing or injection molding that makes the part thicker than that by sheet-metal processing.

2.3 Distance between Light Emitting Part and Light Receiving Part

FIG. 8 shows a relationship between the distance LD between the light emitting part 150 and the light receiving part 140 and the signal intensity of the detection signal. Here, the signal intensity is intensity of the detection signal of a detection apparatus to which the photodetection unit of the embodiment is applied. For example, when the photodetection unit is applied to a detection apparatus of biological information including pulse wave, which will be described later, the signal intensity is intensity of the biological information detection signal including pulse wave. Further, the distance LD between the light emitting part 150 and the light receiving part 140 is e.g., a distance between the center positions (representative positions) of the light emitting part 150 and the light receiving part 140. For example, when the light receiving part 140 has a rectangular shape (nearly rectangular shape), the position of the light receiving part 140 is the center position of the rectangular shape. Further, when the light emitting part 150 has the above described dome lens 151, the position of the light emitting part 150 is e.g., the center position of the dome lens 151 (the position of the LED chip).

As is clear from FIG. 8, the shorter the distance LD between the light emitting part 150 and the light receiving part 140, the higher the signal intensity of the detection signal and the higher the detection performance including sensitivity. Therefore, the shorter the distance LD between the light emitting part 150 and the light receiving part 140, the more desirable.

In this regard, in the embodiment, as shown in the above described FIGS. 2 and 6, the light shielding member 70 is formed by sheet-metal processing of the metal and the light shielding wall 100 is realized by the metal surface 71 thereof. Therefore, compared to the case where the light shielding member 70 is realized by injection molding, it may be possible to make the thickness of the light shielding wall 100 smaller to e.g., about 0.1 mm. Therefore, it may be possible to make the distance LD between the light emitting part 150 and the light receiving part 140 shorter by the amount of the smaller thickness of the light shielding wall 100, and the detection performance of the detection apparatus may be improved as is clear from FIG. 8.

In this case, as shown in FIG. 8, it is desirable that the distance between the light emitting part 150 and the light receiving part 140 is LD<3 mm. For example, as is clear from a tangent line G2 at the larger distance side on a characteristic curve G1 in FIG. 8, in a range of LD>=3 mm, the characteristic curve G1 is saturated. On the other hand, in a range of LD<3 mm, the shorter the distance LD, the more largely the signal intensity increases. Therefore, in this regard, LD<3 mm is desirable.

Further, regarding the distance LD, LD<2.5 mm is desirable. For example, as is understood from the relationship between the tangent line G2 at the larger distance side and a tangent line G3 at the smaller distance side, the increase rate of the signal intensity with respect to the distance is even higher in a distance range of LD<2.5 mm (2.4 mm). Therefore, in this regard, LD<2.5 mm is more desirable.

Furthermore, in the photodetection unit of the embodiment shown in FIGS. 2 and 6, for example, the distance LD is substantially LD=2.0 mm. Therefore, as shown in FIG. 8, compared to the photodetection unit in related art in which LD>=3 mm, the detection performance may be significantly improved.

In addition, regarding the distance LD, there is a lower limit and it is not desirable that distance LD is too short. For example, FIG. 9 shows the case where the photodetection unit of the embodiment is applied to the detection apparatus of biological information including pulse wave. In this case, the light from the light emitting part 150 is diffused or scattered in a vessel or the like of the subject, and the light enters the light receiving part 140 and the pulse wave is detected. In FIG. 9, a relation of LD=2*LB generally holds between the distance LD between the light emitting part 150 and the light receiving part 140 and a measured distance LB in the depth direction. For example, the measurement limit distance by the photodetection unit including the light emitting part 150 and the light receiving part 140 apart at the distance LD is substantially LB=LD/2. In a range of the distance LB, e.g., from 100 micrometers to 150 micrometers, there is no vessel as an object to be detected for pulse wave. Therefore, if the distance LD is LD<=2*LB=2*100 micrometers to 2*150 micrometers=0.2 mm to 0.3 mm, it is expected that the detection signal of pulse wave is extremely smaller. That is, when the distance LD is shorter, the measured distance LB in the depth direction is also shorter, and, if no object to be detected exists in the range of the distance LB, the detection signal becomes extremely smaller. Briefly, the shorter the distance LD, the higher the detection performance, but there is the limitation, and the lower limit exists. Therefore, in this regard, LD>0.3 mm is desirable. Namely, 0.3 mm<LD<2.5 mm (or 0.3 mm<LD<3.0 mm) is desirable.

### 3. Second Embodiment

3.1 Photodetection Unit

As described above using FIG. 1D, the photodetection unit of the embodiment includes the light receiving part 140, the light emitting part 150, the first substrate 161 on which the light emitting part 150 is mounted, and the second substrate 162 on which the light receiving part 140 is mounted. Note that, in the embodiment, the same configurations as those of the first embodiment will be appropriately omitted.

As described above using FIG. 15, the relative position relationships of the respective parts of the photodetection unit of the embodiment are defined by the height h4 from the reference surface to the first substrate 161 and the height h5 from the reference surface to the second substrate 162. Note that the conditions defining the position relationships are not limited to those of h4 and h5, but the position relationships may be defined using other conditions.

For example, as shown in FIG. 15, supposing that the height from the reference surface to the light emitting part 150 is h6 and the height from the reference surface to the light receiving part is h7, the relative position relationships of the respective parts of the photodetection unit may be determined so that h7>h6.

Here, h6 may be considered as a distance from the reference surface to a given reference point of the light emitting part 150 and h7 may be considered as a distance from the reference surface to a given reference point of the light receiving part 140. Note that, if the role as the light shielding member that shields direct light is provided to the second substrate 162, the given reference point of the light emitting part 150 may be set to a position of the light emitting part 150 to which light is applied and the given reference point of the light receiving part 140 may be set to a position of the light receiving part 140 in which light is received.

According to the configuration, as shown in FIG. 15, it may be possible to provide the light emitting part 150 in a lower position (below in FIG. 15) than that of the light receiving part 140 with respect to the reference surface. Accordingly, a condition that the direct light from the light emitting part 150 to the light receiving part 140 is shielded by the second substrate 162 is easily satisfied. Particularly, as described above, h6 and h7 are set with reference to the application point and receiving point of lights, and thereby, the direct light inevitably travels in a direction from the lower position to the higher position (in a direction from downside to upside in FIG. 16 etc.). In this case, it is highly likely that the direct light is shielded by the second substrate 162 in the lower position than that of the light receiving part 140, and the possibility that the direct light is problematic may be suppressed without providing the light shielding wall 100.

As described above using FIG. 3, the lens part 151 is provided in the light emitting part 150, and thereby, improvement of optical performance of the photodetection unit may be expected. Accordingly, as shown in FIG. 1D etc., the light emitting part 150 may have the lens part 151 that condenses light, the enclosing part 153 that encloses the light emitting device, and the base part 155 as the base of the enclosing part 153. In this case, the height h6 of the light emitting part 150 may be set to the height of the lens part 151 from the reference surface.

As described above, the position within the light emitting part 150 determining h6 may be set to the point to which the light is applied. Further, in the light emitting part 150 in which the lens part 151 is provided, the point to which the light is applied is the lens part 151, thus, the height h6 of the light emitting part 150 is set to the height of the lens part 151 from the reference surface, and thereby, it may be possible to set the appropriate relative position relationship.

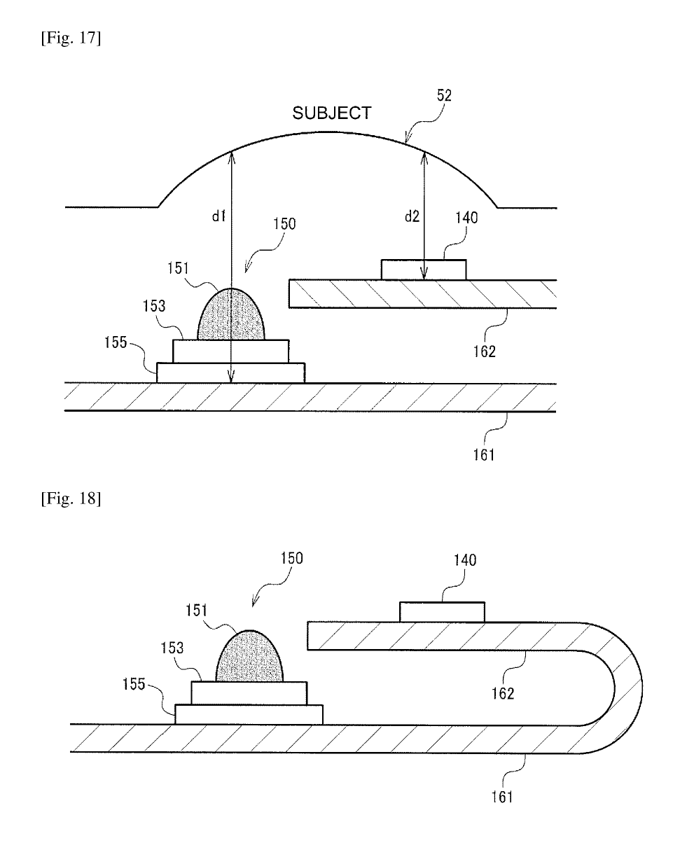

Or, the relative position relationships of the respective parts of the photodetection unit may be defined in a different viewpoint from the height from the reference surface. For example, as shown in FIG. 17, in a state in which an apparatus including the photodetection unit is attached to a subject as the object, supposing that the distance from the first substrate 161 to the object is d1 and the distance from the second substrate 162 to the object is d2, a position relationship d1>d2 may be used.

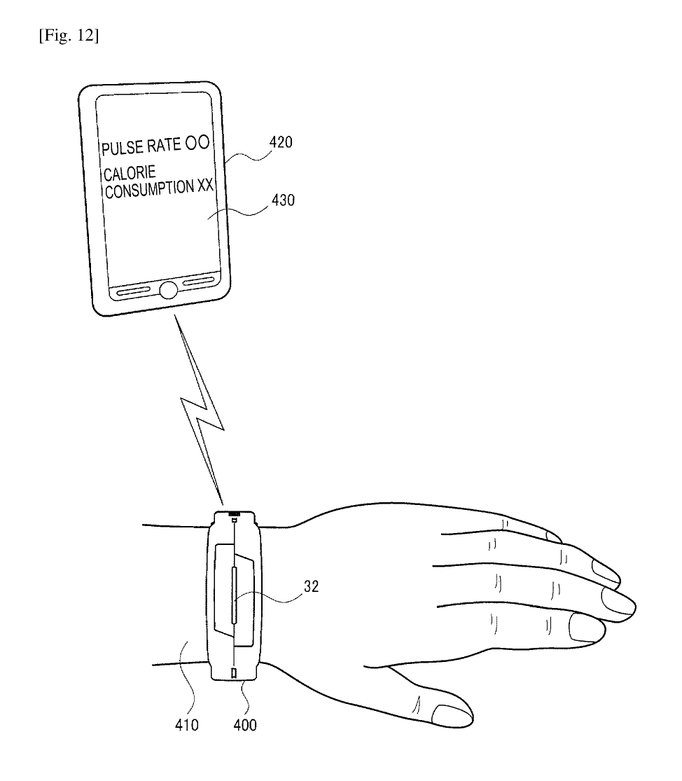

Here, as the apparatus including the photodetection unit, a biological information detection apparatus, which will be described later using FIG. 10A etc. will be explained as an example. In this case, a state in which the apparatus is attached to a subject corresponds to a state in which the apparatus is fixed to a wrist of a user using a band part 10 as shown in FIG. 12.

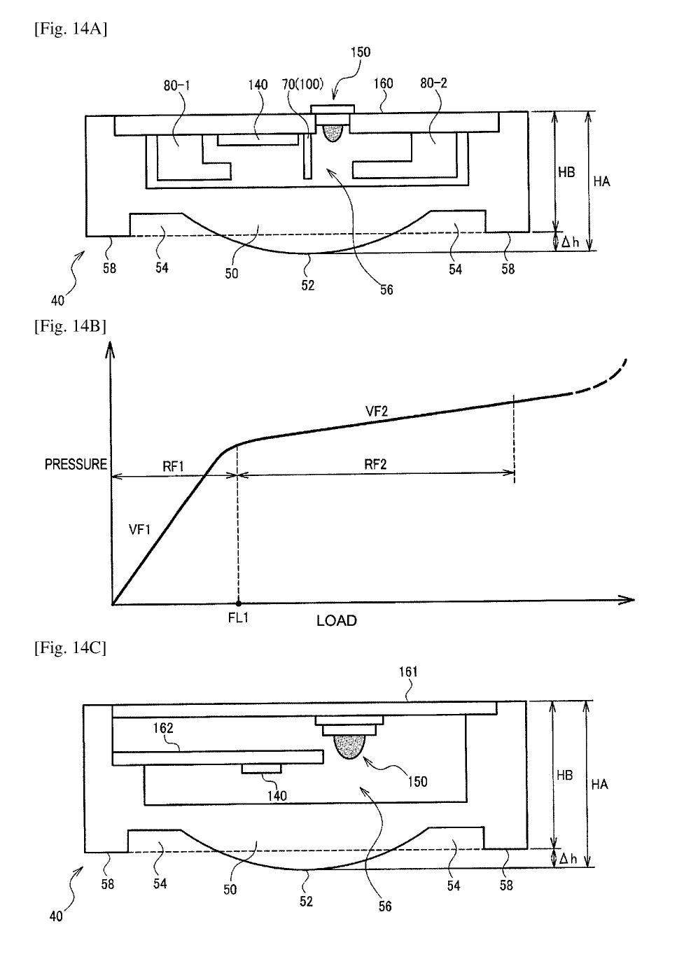

As will be described later using FIG. 14C, the biological information detection apparatus in FIG. 10A applies appropriate pressure to the subject when a convex portion 52 formed by a light-transmissive member 50 is pressed against the subject. That is, in the attached state, the position relationship between the photodetection unit and the subject is as shown in FIG. 17. Here, various techniques of setting the distance d1 between the first substrate 161 and the subject are conceivable. As an example, as shown in FIG. 17, the distance from the point of the first substrate 161 at which the light emitting part 150 is mounted to the point at which a straight line along a direction perpendicular to the first substrate 161 intersects with the subject (the boundary between the convex portion 52 and the subject) may be used as d1. Similarly, the distance from the point of the second substrate 162 at which the light receiving part 140 is mounted to the point at which a straight line along a direction perpendicular to the second substrate 162 intersects with the subject may be used as d2. Note that various modifications may be made regarding the contact condition between the biological information detection apparatus and the subject and the setting condition of d1 and d2.

As is clear from FIG. 17, if the distances d1 and d2 in the attached state are used, it may be possible to provide the difference between the heights of the first substrate 161 and the second substrate 162 (positions in the direction perpendicular to the substrates).

Further, the first substrate 161 and the second substrate 162 may be different two substrates, but not limited to those. For example, as shown in FIG. 18, the first substrate 161 and the second substrate 162 may be integrally formed by a flexible board.

According to the configuration, the difference in height may be provided between the light emitting part 150 and the light receiving part 140 using one substrate. The substrate may be a flexible board bendable as a whole or a rigid-flexible board formed by integrating a rigid board and a flexible board. In the case where the rigid-flexible board is used, use of a structure in which a bendable flexible part is provided between a first rigid part in which the light emitting part 150 is provided and a second rigid part in which the light receiving part 140 is provided is conceivable.

3.2 Distance between Light Emitting Part and Light Receiving Part

The distance between light emitting part and light receiving part is the same as that of the first embodiment. Further, in the photodetection unit of the embodiment shown in FIG. 1D etc., for example, the distance LD is substantially LD=2.0 mm. Therefore, as shown in FIG. 8, compared to the photodetection unit in related art in which LD>=3 mm, the detection performance may be significantly improved.

Furthermore, in the embodiment, the direct light may be shielded by the second substrate 162. Therefore, the distance LD between the light emitting part 150 and the light receiving part 140 may be made shorter by the amount without providing the light shielding wall 100, and, as is clear from FIG. 8, the detection performance of the detection apparatus may be improved.

However, the signal detected in the light receiving part 140 contains various kinds of noise including body motion noise due to body motion etc., and it is preferable to reduce the noise in order to perform processing with higher accuracy. Here, as described above, in the range of LD<0.3 mm or LD>3.0 mm, noise signals are detected by a second light receiving part 141 different from the light receiving part 140 by utilizing detection of information of epidermal or hypodermal tissues and impossibility of acquisition of information on a desired vessel.

Specifically, in the light receiving part 140, 0.3 mm<LD<2.5 mm (or 0.3 mm<LD<3.0 mm) is set and the signals due to the desired vessel are acquired, and, in the second light receiving part 141, LD<0.3 mm or LD>3.0 mm is set and the noise signals are acquired. In this manner, of the signal components detected by the light receiving part 140, the signal components also detected by the second light receiving part 141 may be determined as noise components that should not be detected. Therefore, it may be possible to perform noise reduction processing by removing the noise components from the detection signals of the light receiving part 140.

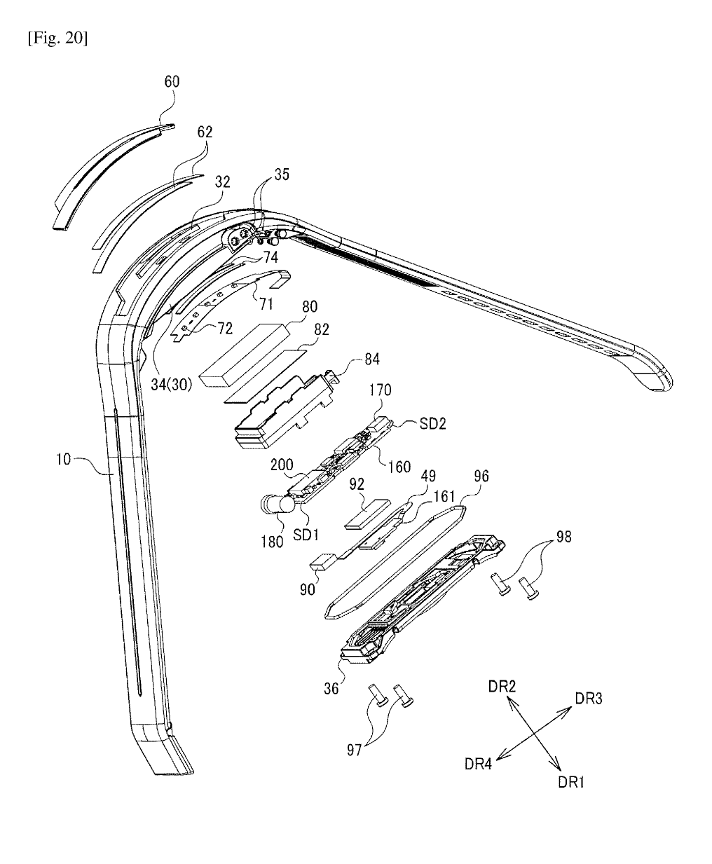

Various techniques are conceivable for the placement of the second light receiving part 141 in this case. For example, in an example shown in FIG. 19A, the photodetection unit further includes the second light receiving part 141 that outputs detection signals for noise reduction and the second light receiving part 141 is mounted on the second substrate 162.

In this regard, as shown in FIG. 19A, supposing that the distance between the light emitting part 150 and the light receiving part 140 is L1 and the distance between the light emitting part 150 and the second light receiving part 141 is L2, L1<L2 may hold. Here, L1 and L2 may be a direct distance between the light emitting part 150 and the light receiving part 140 or the second light receiving part 141, or, as shown in FIG. 19A, a distance of projection on the first surface (or a surface in parallel thereto).

In the case where the sensitivity of detection signals in the light receiving part 140 that detects pulse components is made higher, it is preferable that the distance from the light emitting part 150 to the light receiving part 140 satisfies the above described conditions and is made as small as possible to make the optical path length shorter. In this case, if L2<L1 is set, it is likely that the second light receiving part 141 is mounted between the light emitting part 150 and the light receiving part 140, and it is difficult to make the distance L1 between the light emitting part 150 and the light receiving part 140 smaller. Therefore, in the case where a plurality of light receiving parts are provided on the second substrate 162, L1<L2 may be set as shown in FIG. 19A.

Note that, if the information of a vessel is acquired principally by the light receiving part 140 and the noise components are detected by the second light receiving part 141, as long as the conditions of LD are considered, the magnitude relationship is not limited to L1<L2. For example, the second light receiving part 141 may be provided in a position on the second substrate 162 closer to the light emitting part 150 than the light receiving part 140 (L2<L1).

Further, as shown in FIG. 19B, the photodetection unit may further include the second light receiving part 141 that outputs detection signals for noise reduction and the second light receiving part 141 may be mounted on the first substrate 161.

L2<L1 is set in FIG. 19B, however, as is the case in FIG. 19A, a modification of L2>L1 is possible. Note that, in the case of FIG. 19A, both the light receiving part 140 and the second light receiving part 141 are mounted on the second substrate 162, and thereby, it may be possible to shield both the direct light from the light emitting part 150 to the light receiving part 140 and the direct light from the light emitting part 150 to the second light receiving part 141 and it is unnecessary to provide the light shielding wall 100. However, in FIG. 19B, the second light receiving part 141 is mounted on the first substrate 161, and it is difficult that the second substrate 162 shields the direct light from the light emitting part 150 to the second light receiving part 141. Therefore, as shown in FIG. 19B, the light shielding member 70 (light shielding wall 100) that shields the direct light from the light emitting part 150 to the second light receiving part 141 may be separately provided.

### 4. Biological Information Detection Apparatus

4.1 Overall Configuration Example of Biological Information Detection Apparatus

FIGS. 10A, 10B, and 11 show external views of a biological information detection apparatus of the embodiment (biological information measurement apparatus). FIG. 10A shows the biological information detection apparatus as seen from the front side, FIG. 10B as is seen from the upside, and FIG. 11 is as seen from the side surface side.

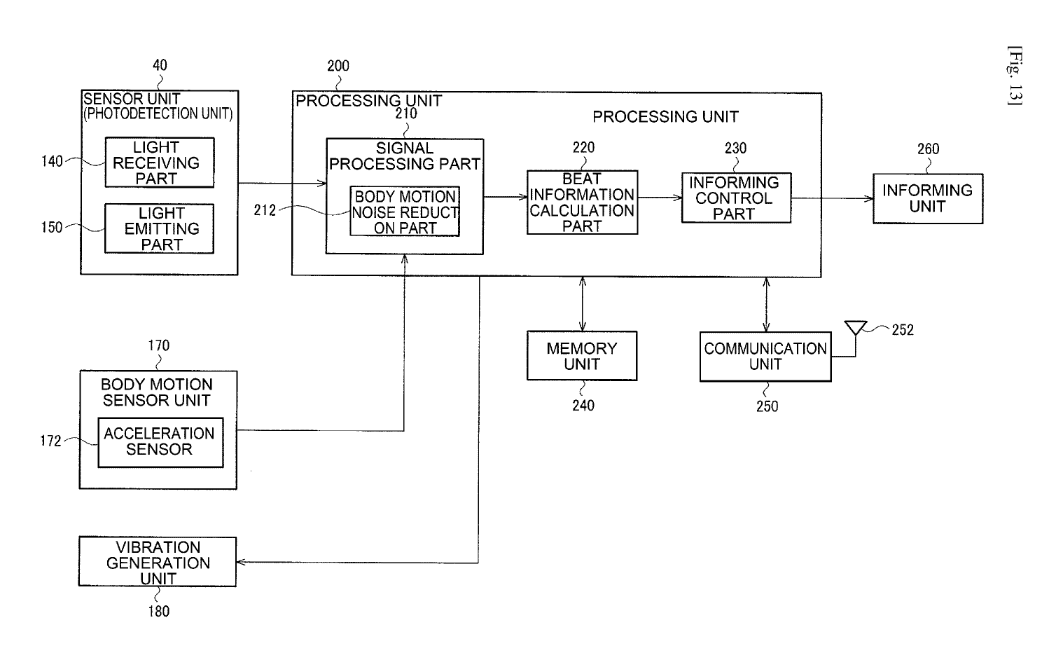

As shown in FIGS. 10A to 11, the biological information detection apparatus of the embodiment has the band part 10, a case unit 30, and a sensor unit 40. The case unit 30 is attached to the band part 10. The sensor unit 40 is provided in the case unit 30. Further, the biological information detection apparatus has a processing unit 200 as shown in FIG. 13, which will be described later. The processing unit 200 is provided in the case unit 30, and detects biological information abased on detection signals from the sensor unit 40. Note that the biological information detection apparatus of the embodiment is not limited to the configuration in FIGS. 10A to 11, but various modifications by omitting part of the component elements, replacing the component elements by other component elements, or adding other component elements may be made.

The sensor unit 40 contains the above described photodetection unit. For example, as will be described later using FIG. 14A, the sensor unit 40 includes the substrate 160, the light emitting part 150, the light receiving part 140, the light shielding member 70, and the photodetection unit having the diaphragm part 80 (80-1, 80-2), and other members. In the example of FIG. 14A, the other members include the convex portion 52, groove portions 54, a concave portion 56, pressure suppression portions 58 realized by the light-transmissive member 50 etc. Note that a modification that the photodetection unit according to the embodiment includes those members, i.e., the whole sensor unit 40 corresponds to the photodetection unit or the like may be made. Alternatively, as will be described later using FIG. 14C, the sensor unit 40 may include the photodetection unit having the first substrate 161, the second substrate 162, the light emitting part 150, and the light receiving part 140 and other members.

The band part 10 is wrapped around the wrist of the user for attachment of the biological information detection apparatus. The band part 10 has band holes 12 and a buckle part 14. The buckle part 14 has a band insertion portion 15 and a projection portion 16. The user inserts one end side of the band part 10 into the band insertion portion 15 of the buckle part 14 and inserts the projection portion 16 of the buckle part 14 into the band hole 12 of the band part 10, and thereby, attaches the biological information detection apparatus to the wrist. In this case, the magnitude of the pressure of the sensor unit 40 (pressure on the wrist surface), which will be described later, is adjusted depending on the band hole 12 into which the projection portion 16 is inserted.

The case unit 30 corresponds to a main body unit of the biological information detection apparatus. Within the case unit 30, various component parts of the biological information detection apparatus including the sensor unit 40 and the processing unit 200 are provided. That is, the case unit 30 is a casing housing the component parts. The case unit 30 has e.g., a top case 34 and a bottom case 36. Note that the case unit 30 does not necessary have the form separated into the top case 34 and the bottom case 36.

A light emitting window part 32 is provided in the case unit 30. The light emitting window part 32 is formed by the light-transmissive member. Further, in the case unit 30, a light emitting part (LED, a light emitting part for informing different from the light emitting part 150 of the photodetection unit) mounted on a flexible board is provided, and light from the light emitting part is output to the outside of the case unit 30 via the light emitting window part 32.

As shown in FIG. 11, terminal portions 35 are provided in the case unit 30. When the biological information detection apparatus is attached to a cradle (not shown), the terminal portions of the cradle and the terminal portions 35 of the case unit 30 are electrically connected. Thereby, it may be possible to recharge a secondary cell (battery) provided in the case unit 30.

The sensor unit 40 detects biological information including pulse wave of the subject. For example, the sensor unit 40 has the light receiving part 140 and the light emitting part 150 as shown in FIGS. 13, 14A, and 14C, which will be described later. Further, the sensor unit 40 has the convex portion 52 formed by the light-transmissive member and coming into contact with the skin surface of the subject and applying pressure thereto. In the state in which the convex portion 52 applies pressure to the skin surface, the light emitting part 150 emits light and the light reflected by the subject (vessel) is received by the light receiving part 140, and the result of received light is output to the processing unit 200 as a detection signal. Then, the processing unit 200 detects biological information including pulse wave based on the detection signal from the sensor unit 40. The biological information as an object to be detected of the biological information detection apparatus of the embodiment is not limited to pulse wave (pulse rate), but the biological information detection apparatus may be an apparatus that detects other biological information than pulse wave (e.g., oxygen saturation in blood, body temperature, heart rate, or the like).

FIG. 12 is an explanatory diagram of attachment of a biological information detection apparatus 400 and communication with a terminal device 420.

As shown in FIG. 12, the user as the subject wears the biological information detection apparatus 400 on a wrist 410 like a wristwatch. As shown in FIG. 11, the sensor unit 40 is provided on the surface at the subject side of the case unit 30. Therefore, when the biological information detection apparatus 400 is attached, the convex portion 52 of the sensor unit 40 comes into contact with the skin surface of the wrist 410 and applies pressure thereto, in the state, the light emitting part 150 of the sensor unit 40 emits light and the light receiving part 140 receives reflected light, and thereby, biological information including pulse wave is detected.

The biological information detection apparatus 400 and the terminal device 420 are communication-connected to enable transmission and reception of data. The terminal device 420 is a portable communication terminal such as a smartphone, a cell phone, or a future phone. Alternatively, the terminal device 420 may be an information processing terminal such as a tablet computer. As the communication connection between the biological information detection apparatus 400 and the terminal device 420, near-field wireless communication, e.g., Bluetooth (registered trademark) or the like may be employed.

The biological information detection apparatus 400 and the terminal device 420 are thus communication-connected, and thereby, various kinds of information including pulse rate and calorie consumption may be displayed on a display unit 430 (LCD or the like) of the terminal device 420. That is, various kinds of information obtained based on the detection signals of the sensor unit 40 may be displayed. Note that calculation processing of the information including pulse rate and calorie consumption may be executed in the biological information detection apparatus 400 or at least part of the processing may be executed in the terminal device 420.

In the biological information detection apparatus 400, the light emitting window part 32 is provided and informs the user of various kinds of information by emitting light (lighting, blinking) of the light emitting part for informing. For example, at entrance to a fat-burning zone and exit from the fat-burning zone, the user is informed by emission of the light emitting part via the light emitting window part 32. Further, when a mail or the like is received in the terminal device 420, this is reported to the biological information detection apparatus 400 from the terminal device 420. Then, the light emitting part of the biological information detection apparatus 400 emits light, and thereby, the user is informed of reception of the mail or the like.

As described above, in FIG. 12, the display unit such as an LCD is not provided in the biological information detection apparatus 400, and the information necessary for informing using characters, numerals, etc. is displayed on the display unit 430 of the terminal device 420. In FIG. 12, the user is informed of the minimum information by light emission of the light emitting part without provision of the display unit such as an LCD, and thereby, downsizing of the biological information detection apparatus 400 is realized. The display unit is not provided in the biological information detection apparatus 400, and thereby, also the aesthetic design of the biological information detection apparatus 400 may be improved.

FIG. 13 shows a functional block diagram of the biological information detection apparatus of the embodiment. In FIG. 13, the biological information detection apparatus includes the sensor unit 40, a body motion sensor unit 170, a vibration generation unit 180, the processing unit 200, a memory unit 240, a communication unit 250, an antenna 252, and an informing unit 260. Note that the biological information detection apparatus of the embodiment is not limited to the configuration in FIG. 13, but various modifications by omitting part of the component elements, replacing the component elements by other component elements, or adding other component elements may be made.

The sensor unit 40 detects biological information including pulse wave, and includes the light receiving part 140 and the light emitting part 150. These light receiving part 140, light emitting part 150, etc. realize a pulse wave sensor (photoelectric sensor). The sensor unit 40 outputs signals detected by the pulse wave sensor as pulse wave detection signals.

The body motion sensor unit 170 outputs body motion detection signals as signals that change in response to body motion based on sensor information of various sensors. The body motion sensor unit 170 includes e.g., an acceleration sensor 172 as a body motion sensor. Note that the body motion sensor unit 170 may have a pressure sensor or gyro sensor as the body motion sensor.

The processing unit 200 performs various kinds of signal processing and control processing using e.g., the memory unit 240 as a work area and is realized by e.g., a processor such as a CPU or a logic circuit such as an ASIC. The processing unit 200 includes a signal processing part 210, a beat information calculation part 220, and an informing control part 230.

The signal processing part 210 performs various kinds of signal processing (filter processing etc.) and performs signal processing on e.g., the pulse wave detection signals from the sensor unit 40 and the body motion detection signals from the body motion sensor unit 170. For example, the signal processing part 210 includes a body motion noise reduction part 212. The body motion noise reduction part 212 performs processing of reducing (removing) body motion noise as noise due to body motion from the pulse wave detection signals based on the body motion detection signals from the body motion sensor unit 170. Specifically, the part performs noise reduction processing using e.g., an adaptive filter or the like.

The beat information calculation part 220 performs calculation processing of beat information based on the signals from the signal processing part 210 or the like. The beat information is e.g., information including pulse rate. Specifically, the beat information calculation part 220 performs frequency analysis processing such as FFT on the pulse wave detection signals after the noise reduction processing in the body motion noise reduction part 212 to obtain a spectrum, and performs processing of using the representative frequency in the obtained spectrum as a frequency of heartbeat. A value by sixty-fold of the obtained frequency is the pulse rate (heart rate) generally used. Note that the beat information is not limited to the pulse rate itself, but may be other various kinds of information representing the pulse rate (e.g., frequency or period of heartbeat or the like). Or, the beat information may be information representing a beating status, and e.g., a value representing the blood volume itself may be used as the beat information.

The informing control part 230 controls the informing unit 260. The informing unit 260 (informing device) informs the user of various kinds of information under control of the informing control part 230. As the informing unit 260, e.g., the light emitting part for informing may be used. In this case, the informing control part 230 controls the current flowing in the LED, and thereby, controls lighting, blinking, or the like of the light emitting part. The informing unit 260 may be a display unit such as an LCD, a buzzer, or the like.

Further, the informing control part 230 controls the vibration generation unit 180. The vibration generation unit 180 informs the user of various kinds of information by vibration. The vibration generation unit 180 may be realized by e.g., a vibration motor (vibrator). The vibration motor generates vibration by rotating e.g., an eccentric weight. Specifically, eccentric weights are attached to both ends of a drive shaft (rotor shaft) so that the motor itself may swing. The vibration of the vibration generation unit 180 is controlled by the informing control part 230. Note that the vibration generation unit 180 is not limited to the vibration motor, but various modifications may be made. For example, the vibration generation unit 180 may be realized using a piezoelectric element.

The vibration by the vibration generation unit 180 enables e.g., informing of startup when the power is turned on, informing of successful first pulse wave detection, warning when a state of being impossible to detect pulse wave continues in a fixed period, informing at movement of the fat-burning zone, warning at battery voltage reduction, notification of wakeup alarm, or reporting of mails and calls from a terminal device such as a smartphone. The user may be informed of the information by the light emitting part for informing or by both the vibration generation unit 180 and the light emitting part.

The communication unit 250 performs communication processing with the external terminal device 420 as explained in FIG. 12. For example, the unit performs processing of wireless communication according to the standard such as Bluetooth (registered trademark). Specifically, the communication unit 250 performs reception processing of signals from the antenna 252 and transmission processing to the antenna 252. The function of the communication unit 250 may be realized by a processor for communication or a logic circuit such as an ASIC.

4.2 Configuration Example of Sensor Unit

FIG. 14A shows a detailed configuration example of the sensor unit 40. The sensor unit 40 has the light receiving part 140 and light emitting part 150. These light receiving part 140 and the light emitting part 150 are mounted on the substrate 160 (sensor board). The light receiving part 140 receives light from the subject (reflected light, transmitted light, or the like). The light emitting part 150 outputs light to the subject. For example, when the light emitting part 150 outputs light to the subject and the light is reflected by the subject (vessel), the light receiving part 140 receives and detects the reflected light. The light receiving part 140 may be realized using a light receiving device such as e.g. a photodiode. The light emitting part 150 may be realized using a light emitting device such as e.g. an LED. For example, the light receiving part 140 may be realized using a P-N junction diode device formed on a semiconductor substrate or the like. In this case, an angle-limiting filter that narrows down the light receiving angle and a wavelength-limiting filter that limits the wavelength of light entering the light receiving device may be formed on the diode device.

When the pulsimeter is taken as an example, the light from the light emitting part 150 travels inside of the subject and disperses or scatters in epidermal, dermal, hypodermal tissues or the like. Then, the light reaches a vessel (part to be detected) and is reflected. In this regard, part of the light is absorbed by the vessel. Then, the absorptance of the light in the vessel changes due to pulse and the amount of reflected light changes. The light receiving part 140 receives the reflected light and detects the changes of the amount of light, and thereby, the pulse rate etc. as biological information may be detected.

The light shielding member 70 (light shielding wall 100) is provided between the light receiving part 140 and the light emitting part 150. For example, the light shielding member 70 shields the light from the light emitting part 150 directly entering the light receiving part 140.

Further, the diaphragm part 80 (80-1, 80-2) is provided in the sensor unit 40. The diaphragm part 80 narrows down the light from the subject and narrows down the light from the light emitting part 150 in the optical path between the subject and the sensor unit 40. In FIG. 14A, the diaphragm part 80 is provided between the light-transmissive member 50 and the sensor unit 40. Note that the diaphragm part 80 may be provided between the light-transmissive member 50 and the subject or within the light-transmissive member 50. Further, the light shielding member 70 and the diaphragm part 80 may be integrally formed by sheet-metal processing of a metal, for example.

The light-transmissive member 50 is provided on the surface at the side of the biological information detection apparatus in contact with the subject, and transmits the light from the subject. Further, the light-transmissive member 50 comes into contact with the subject at measurement of biological information of the subject. For example, the convex portion 52 (detection window) of the light-transmissive member 50 comes into contact with the subject. Note that the surface shape of the convex portion 52 is desirably a curved shape (spherical shape), however, various shapes may be employed, not limited to that. Further, as long as the light-transmissive member 50 is transparent with respect to the wavelength of the light from the subject, a transparent material may be used or a colored material may be used.

Around the convex portion 52 of the light-transmissive member 50, the groove portions 54 for suppressing pressure variations or the like are provided. Further, in the case where the surface at the side at which the convex portion 52 is provided in the light-transmissive member 50 is the first surface, the light-transmissive member 50 has the concave portion 56 in the position corresponding to the convex portion 52 on the second surface at the rear side of the first surface. In the space of the concave portion 56, the light receiving part 140, the light emitting part 150, the light shielding member 70, and the diaphragm part 80 are provided.

On the surface at the subject side of the biological information detection apparatus, the pressure suppression portions 58 that suppresses the pressure that the convex portion 52 applies to the subject (the skin of the wrist) is provided. In FIG. 14A, the pressure suppression portions 58 is provided to surround the convex portion 52 of the light-transmissive member 50.

Further, in FIG. 14A, supposing that the height of the convex portion 52 in the direction orthogonal to the surface at the subject side of the biological information detection apparatus is HA (e.g., the height of the vertex of the curved shape of the convex portion 52), the height of the pressure suppression portions 58 is HB (e.g., the height of the highest location), and a value obtained by subtracting the height HB from the height HA (a difference between the heights HA and HB) is delta_h, a relation delta_h=HA−HB>0 holds. For example, the convex portion 52 projects from the pressure suppression portions 58 toward the subject side so that delta_h>0 may be satisfied. That is, the convex portion 52 projects from the pressure suppression portions (pressure suppression surfaces) 58 toward the subject side by the amount of delta_h.

As described above, the convex portion 52 that satisfies delta_h>0 is provided, and thereby, it may be possible to apply e.g., initial pressure for exceeding a vein disappearance point to the subject. Further, the pressure suppression portions 58 that suppress the pressure that the convex portion 52 applies to the subject are provided, and thereby, in a use range in which measurement of the biological information is performed by the biological information detection apparatus, the pressure variations may be minimized and noise components or the like may be reduced. Furthermore, when the convex portion 52 projects from the pressure suppression portions 58 so that delta_h>0 may be satisfied, the convex portion 52 comes into contact with the subject and applies the initial pressure thereto, then, the pressure suppression portions 58 come into contact with the subject, and thereby, the pressure that the convex portion 52 applies to the subject may be suppressed. Here, the vein disappearance point is a point at which, when the convex portion 52 is brought into contact with the subject and the pressure is gradually made stronger, the signal due to the vein superimposed on the pulse wave signal disappears or becomes smaller to the degree not affecting the pulse wave measurement.

For example, in FIG. 14B, the horizontal axis indicates load generated by a load mechanism (a mechanism including the band part, the buckle part, etc.) of the biological information detection apparatus, and the vertical axis indicates pressure applied by the convex portion 52 to the subject (pressure applied to the vessel). Suppose that the amount of change in pressure of the convex portion 52 with respect to the load mechanism generating the pressure of the convex portion 52 is the amount of pressure change. The amount of pressure change corresponds to the gradient of the change characteristic of the pressure with respect to the load.

In this case, the pressure suppression portions 58 suppress the pressure applied by the convex portion 52 to the subject so that, with respect to an amount of pressure change VF1 in a first load range RF1 in which the load of the load mechanism is from zero to FL1, an amount of pressure change VF2 in a second load range RF2 in which the load of the load mechanism is larger than FL1 may be smaller. That is, the amount of pressure change VF1 is made larger in the first load range RF1 as an initial pressure range, and the amount of pressure change VF2 is made smaller in the second load range RF2 as the use range of the biological information detection apparatus.

Namely, in the first load range RF1, the amount of pressure change VF1 is made larger and the gradient of the change characteristic of the pressure with respect to the load is made larger. The pressure with the larger gradient of the change characteristic is realized by delta_h corresponding to the amount of projection of the convex portion 52. That is, the convex portion 52 that satisfies delta_h>0 is provided, and thereby, even when the load by the load mechanism is smaller, it may be possible to apply the necessary and sufficient initial pressure to exceed the vein disappearance point to the subject.

On the other hand, in the second load range RF2, the amount of pressure change VF2 is made smaller and the gradient of the change characteristic of the pressure with respect to the load is made smaller. The pressure with the smaller gradient of the change characteristic is realized by pressure suppression by the pressure suppression portions 58. That is, the pressure applied by the convex portion 52 to the subject is suppressed by the pressure suppression portions 58, and thereby, in the use range of the biological information detection apparatus, even in the case where load varies or the like, it may be possible to minimize the pressure variations. Thereby, reduction of noise components or the like may be realized.

As described above, the optimized pressure (e.g., about 16 kPa) is applied to the subject, and thereby, it may be possible to obtain a pulse wave detection signal with a higher M/N ratio (S/N ratio). That is, the signal component of the pulse wave sensor may be increased and the noise component may be reduced. Here, M represents the signal level of the pulse wave detection signal and N represents the noise level.

Further, the pressure range used for pulse wave measurement is set to the range corresponding to the second load range RF2, and thereby, it may be possible to suppress the minimum pressure variations (e.g., about +/−4 kPa) and the noise component may be reduced.

Furthermore, as described above in the second embodiment, the sensor unit 40 may have a configuration corresponding to FIG. 1D. FIG. 14C shows the detailed configuration example of the sensor unit 40. The sensor unit 40 has the light receiving part 140 and the light emitting part 150. The light receiving part 140 is mounted on the second substrate 162 (sensor board) and the light emitting part 150 is mounted on the first substrate 161 (circuit board). The light receiving part 140 receives light from the subject (reflected light, transmitted light, or the like). The light emitting part 150 outputs light to the subject. For example, when the light emitting part 150 outputs light to the subject and the light is reflected by the subject (vessel), the light receiving part 140 receives and detects the reflected light.

4.3 Other Detailed Structure Examples

As described above, the biological information detection apparatus of the embodiment has the band part 10, the case unit 30 attached to the band part 10, the sensor unit 40 provided in the case unit 30, and the processing unit 200 that detects biological information based on the detection signals from the sensor unit 40.

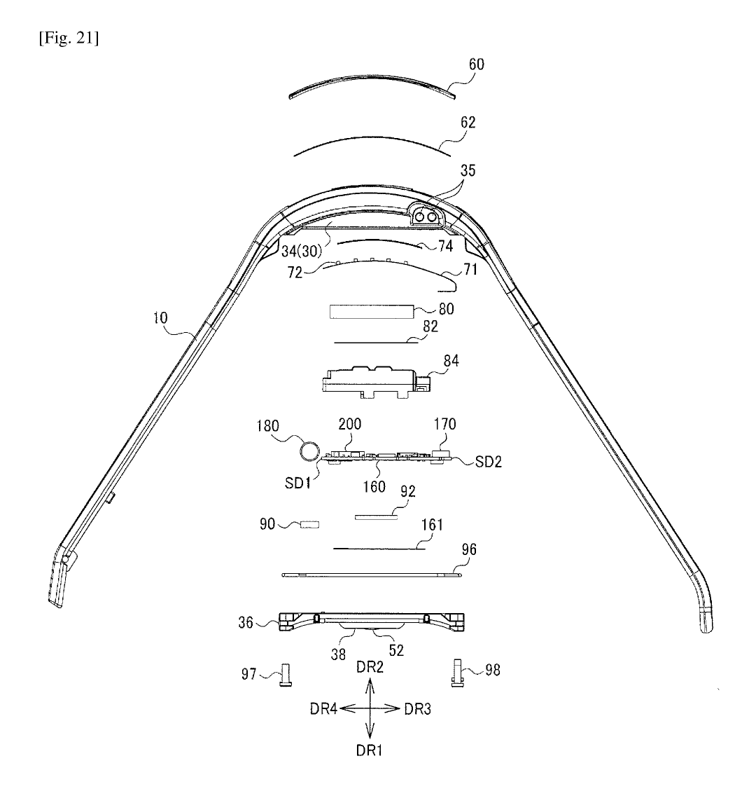

FIGS. 20 and 21 are exploded view showing internal structures etc. of the case unit 30. 60 denotes an ornamental cover and 62 denotes double-sided adhesive tapes. The ornamental cover 60 is bonded to the outer surface of the top case 34 by the double-sided adhesive tapes 62. It may be possible to improve the aesthetic design of the biological information detection apparatus by providing the ornamental cover 60.

71 denotes a flexible board and 74 denotes double-sided adhesive tapes. A light emitting part 72 such as an LED is mounted on the flexible board 71. Further, the antenna 252 is provided on the flexible board 71. Specifically, a metal pattern (not shown) of the antenna 252 is formed on the flexible board 71. Here, the light emitting part 72 is a light emitting part for informing, and different from the light emitting part 150 of the above described photodetection unit. Further, the flexible board 71 is the board on which the light emitting part 72 is mounted, and assumed to be different from either of the above described first substrate 161 or second substrate 162.

80 denotes a secondary cell (battery), 82 denotes a double-sided adhesive tape, and 84 denotes a holder of the secondary cell 80. The secondary cell 80 is bonded to the holder 84 by the double-sided adhesive tape.

160 denotes the circuit board (main board), 170 denotes the body motion sensor unit, 180 denotes the vibration generation unit (vibration motor), and 200 denotes the processing unit. The body motion sensor unit 170 and the processing unit 200 are mounted on the circuit board 160. As is known from the same sign (160) as that of the first substrate assigned to the circuit board, the first substrate 161 of the embodiment may be realized as a main board, for example.

161 denotes the sensor board and 49 denotes a connecting cable. The sensor board is a board on which the light receiving part 140 is mounted and corresponds to the above described second substrate 162. The sensor board 161 and the circuit board 160 are electrically connected by the connecting cable 49. Note that, as described above using FIG. 18, the circuit board 160 and the sensor board 161 may be integrally formed using the flexible board, and, in this case, wiring may be provided on the board. 90, 92, 96 denote buffer members.

36 denotes the bottom case 36, 97 and 98 denote screws. The top case 34 and the bottom case 36 are fastened by the screws 97 and 98.

As shown in FIGS. 20 and 21, the circuit board 160 on which the processing unit 200 is mounted is provided in the case unit 30. The circuit board 160 is e.g., a rigid board. Further, the secondary cell 80 is provided between the circuit board 160 and the top case 34 (the outer surface of the case unit 30). Specifically, the secondary cell 80 is provided between a rear surface 33 of the top case 34 and the circuit board 160, and the rear surface 33 of the top case 34 is a curved surface.

The secondary cell 80 supplies power to the circuit board 160 (processing unit 200, body motion sensor unit 170), the vibration generation unit 180, the sensor unit 40, etc. For example, the biological information detection apparatus is attached to the cradle, and thereby, the terminal portions of the cradle and the case terminal portions 35 are electrically connected and the secondary cell 80 is recharged by the power from the cradle. As the secondary cell 80, e.g., a lithium ion polymer cell or the like may be employed.

In the embodiment, the secondary cell 80 is provided between the circuit board 160 and the top case 34. Therefore, the secondary cell 80 may be provided by effectively utilizing the empty space at the side in the DR2 direction of the circuit board 160. For example, the inner surface 33 of the top case 34 is the curved surface and the relatively large empty space may be secured at the side in the DR2 direction of the circuit board 160. In the embodiment, the secondary cell 80 having a larger volume is provided in the empty space. Thereby, it may be possible to place the parts by effectively utilizing the space within the case unit 30, and reduction in thickness and size of the case unit 30 or the like may be realized.

As above, the embodiments are explained in detail, however, a person skilled in the art could readily understand that many modifications without substantially departing from the new matter and the advantages of the invention may be made. Therefore, those modified examples may fall within the scope of the invention. For example, in the specification or the drawings, the terms described with different terms in broader senses or synonymous at least once may be replaced by the different terms in any part of the specification or the drawings. Further, the configurations and operations of the photodetection unit, the biological information detection apparatus etc. are not limited to those explained in the embodiments, but various modifications may be made.

1. A photodetection unit comprising:
a substrate;
a light emitting part that outputs light to an object; and
a light receiving part attached to the substrate and receiving light from the object,
wherein a hole part is provided in the substrate, and
the light emitting part is attached to the hole part of the substrate.

2. The photodetection unit according to claim 1, wherein the light receiving part is attached to a first surface of the substrate, and the light emitting part is inserted into the hole part from a second surface side of the substrate as a rear surface of the first surface.

3. The photodetection unit according to claim 2, wherein the light emitting part has a lens part that condenses light, and in the light emitting part, the lens part is inserted into the hole part to project toward the first surface side.

4. The photodetection unit according to claim 2, wherein the light emitting part has:
a light emitting device;
an enclosing part in which the light emitting device is enclosed; and
a base part as a base of the enclosing part,
wherein the base part is provided at the second surface side in a state in which the light emitting part is inserted into the hole part.

5. The photodetection unit according to claim 4, wherein the base part has a terminal electrically connected to the light emitting device, and the terminal is electrically connected to a wire provided on the second surface of the substrate.

6. The photodetection unit according to claim 1, wherein a light shielding member that shields at least the light receiving part from light is provided on the substrate.

7. The photodetection unit according to claim 6, wherein the light shielding member has a light shielding wall that shields light from the light emitting part entering the light receiving part, and supposing that a height of the light shielding wall is h1 and a height of the light emitting part is h2, h1>=h2 is satisfied.

8. The photodetection unit according to claim 7, wherein, supposing that a height of the light receiving part is h3, h1>=h2>h3 is satisfied.

9. The photodetection unit according to claim 7, wherein the light shielding member further has a diaphragm part.

10. The photodetection unit according to claim 9, wherein the light shielding wall is formed by sheet-metal processing, and the diaphragm part is formed by the sheet-metal processing or injection molding.

11. A photodetection unit comprising:
a light emitting part that outputs light to an object;
a light receiving part that receives light from the object;
a first substrate on which the light emitting part is mounted; and
a second substrate on which the light receiving part is mounted, wherein, supposing that a height of the first substrate from a reference surface set at an opposite side to the object with respect to the first substrate is h4 and a height of the second substrate from the reference surface is h5, h5>h4 is satisfied.

12. The photodetection unit according to claim 11, wherein the second substrate shields direct light from the light emitting part to the light receiving part.

13. The photodetection unit according to claim 11, wherein, in a state in which an apparatus including the photodetection unit is attached to a subject as the object, supposing that a distance from the first substrate to the object is d1 and a distance from the second substrate to the object is d2, d1>d2 is satisfied.

14. The photodetection unit according to claim 11, wherein, supposing that a height of the light emitting part from the reference surface is h6 and a height of the light receiving part from the reference surface is h7, h7>h6 is satisfied.

15. The photodetection unit according to claim 14, wherein the light emitting part has a lens part that condenses light, an enclosing part in which the light emitting device is enclosed, and a base part as a base of the enclosing part, and
the height h6 of the light emitting part is a height of the lens part from the reference surface.

16. The photodetection unit according to claim 11, further comprising a second light receiving part that outputs a detection signal for noise reduction,
wherein the second light receiving part is mounted on the first substrate.

17. The photodetection unit according to claim 11, further comprising a second light receiving part that outputs a detection signal for noise reduction,
wherein the second light receiving part is mounted on the second substrate.

18. The photodetection unit according to claim 17, wherein, supposing that a distance between the light emitting part and the light receiving part is L1 and a distance between the light emitting part and the second light receiving part is L2, L1<L2 is satisfied.

19. The photodetection unit according to claim 11, wherein the first substrate and the second substrate are integrally formed by a flexible board.

20. The photodetection unit according to claim 11, wherein, supposing that a surface of the first substrate on which the light emitting part is mounted is a first surface, a rear surface of the first surface of the first substrate is a second surface, and a direction from the first surface to the second surface is a first direction,
the reference surface is a surface located at a side in the first direction of the second surface and in parallel to the second surface.

21. A photodetection unit comprising:
a light emitting part that outputs light to an object;
a light receiving part that receives light from the object;
a first substrate on which the light emitting part is mounted; and
a second substrate on which the light receiving part is mounted,
wherein, in a state in which an apparatus including the photodetection unit is attached to a subject as the object, supposing that a distance from the first substrate to the object is d1 and a distance from the second substrate to the object is d2, d1>d2 is satisfied.

22. A biological information detection apparatus comprising the photodetection unit according to claim 1.

23. A biological information detection apparatus comprising the photodetection unit according to claim 11.

24. A biological information detection apparatus comprising the photodetection unit according to claim 21.
